(19) United States
(12) Patent Application Publication
(10) Pub. No.: US 2026/0335893 A1
(43) Pub. Date: Oct. 29, 2026

(54) SYSTEMS, METHODS, AND APPARATUS FOR A MICROFLUIDIC CHIP HAVING A MICROCHANNEL DESIGN WHICH ASYMMETRICALLY FOCUSES PARTICLES

(71) Applicant: ABS Global, Inc., DeForest, WI (US)

(72) Inventors: Wujun Zhao, Waunakee, WI (US); Brian Lena, Sun Prairie, WI (US); Zheng Xia, Middleton, WI (US)

(73) Assignee: ABS Global, Inc., DeForest, WI (US)

(21) Appl. No.: 18/997,222

(22) PCT Filed: Jun. 15, 2023
(86) PCT No.: PCT/US23/68542

§ 371 Date: Jan. 21, 2025

Related U.S. Application Data
(60) Provisional application No. 63/368,988, filed on Jul. 21, 2022.

(51) Int. Cl. B01L 3/00

(52) U.S. Cl. CPC B01L 3/502761; B01L 2200/027; B01L 2200/0636; B01L 2200/0647

(57) ABSTRACT

What is provided is an asymmetrically focusing microfluidic chip and a method of asymmetrically focusing and processing particles using an asymmetrically focusing microfluidic chip. The microfluidic chip comprises a microfluidic channel structure with a sheath fluid channel structure originating upstream at a sheath fluid inlet, the sheath fluid channel structure comprising a first side channel structure and a second side channel structure each having a plurality of tines. A sheath fluid flows through the sheath fluid channel structure into a flow channel at an intersection with a sample fluid flowing from a sample fluid channel. The sample fluid is asymmetrically focused in the intersection. The sample fluid may be analyzed and processed before flowing out of the channel structure via one or more outlets.

### FIELD OF THE INVENTION

The present disclosure relates generally to microfluidic chips and, more specifically, to a microfluidic system and method for optimized focusing and orientation of particles within a microchannel of the microfluidic system. Microfluidic devices and methods for focusing particles in a fluid sample are described herein.

### BACKGROUND

In the separation of various particles or cellular materials—for example, the separation of sperm into viable and motile sperm from non-viable or non-motile sperm, or separation by gender—the process is often a time-consuming task, with severe volume restrictions. Thus, current separation techniques cannot, for example, produce the desired yield, or process volumes of cellular materials in a timely fashion.

Photo-damaging laser systems have utilized lasers to photodamage or kill undesired cellular objects. However, the prior art has required flow cytometers using nozzles, to interrogate and arrange the individual objects in droplet flow, and to attempt to separate and photodamage the objects as they fall into various containers-which has been difficult to achieve.

Thus, there exists a present need for a method and apparatus which identifies and discriminates between target objects, is continuous, has high throughput, is time and cost-effective, and causes negligible or minimal damage to the various target objects. In addition, such an apparatus and method should have further applicability to other biological and medical areas, not just in sperm discrimination, but in the discrimination of blood and other cellular materials, including viral, cell organelle, globular structures, colloidal suspensions, and other biological materials.

A wide range of devices has been introduced for microfluidic sorting of cells and/or microparticles. Specifically, it is often desired to separate various particles or cells from the sample fluid mixture, such as the separation of viable and motile sperm from non-viable and non-motile sperm or the separation of sperm by gender. Precise manipulation of particle position inside microscale flow enables highly efficient sorting of particles, if differential markers exist. Specifically, spatial differentiation of particles or cells can be achieved by taking advantage of hydrodynamic forces due to the physical structure of the microfluidic channel or the intense interaction between particles suspended in flow.

Microfluidics enables the use of small volumes for preparing and processing samples, such as various particles or cellular materials. When separating a sample, such as the separation of sperm into viable and motile sperm from non-viable or non-motile sperm, or separation by gender, the process is often a time-consuming task and can have severe volume restrictions. Current separation techniques cannot, for example, produce the desired yield, or process volumes of cellular materials in a timely fashion. Furthermore, existing microfluidic devices do not effectively focus or orient the sperm cells.

Hence, there is need for a microfluidic device and separation process utilizing said device that is continuous, has high throughput, provides time saving, and causes negligible or minimal damage to the various particles of the separation. In addition, such a device and method can have further applicability to biological and medical areas, not just in sperm sorting, but in the separation of blood and other cellular materials, including viral, cell organelle, globular structures, colloidal suspensions, and other biological materials.

### SUMMARY OF THE INVENTION

What is provided are microfluidic channel structures for use in microfluidic chips and flow cytometry systems which provide for the asymmetric focusing of a sample fluid stream. Through the asymmetric introduction of a sheath or buffer fluid from opposing sets of corresponding tines or “bones” in a structure which resembles a herringbone pattern, a sample fluid stream is hydrodynamically focused in an asymmetric manner.

In accordance with the principles of the present disclosure, a microfluidic chip comprises an asymmetrically focusing microchannel structure comprising opposing, corresponding sets of tines. The tines introduce a sheath or buffer fluid into a flow channel in which a sample fluid has already been flowed or has been previously introduced. The asymmetric introduction of the sheath or buffer fluids into the flow channel progressively compresses, narrows, directs, or focuses the sample fluid stream into a relatively narrower stream while maintaining a laminar or sheath flow. The asymmetric action on the sample fluid stream further aligns, spaces, or orients particles within the sample fluid stream for interrogation, inspection, or action.

Provided herein are systems and methods for the asymmetric focusing of a sample fluid in a microfluidic chip for the interrogation, analysis, and action on the sample fluid.

In one embodiment, what is provided is a microfluidic channel structure comprising: a sheath fluid channel structure originating upstream at a sheath fluid inlet, the sheath fluid channel structure comprising a first side channel structure and a second side channel structure; a sample fluid channel originating at an upstream sample fluid inlet; a flow channel disposed downstream of the sample fluid inlet and the sheath fluid inlet; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In various embodiments, the intersection of the plurality of tines with the flow channel is after a sample introduction location.

In various embodiments, the intersection of the plurality of tines with the flow channel is staggered.

In various embodiments, the intersection of the plurality of tines with the flow channel alternates between an intersection of a tine from the first side channel structure and an intersection of a tine from the second side channel structure.

In various embodiments, the plurality of tines comprises a set of four tines.

In various embodiments, the plurality of tines intersects the flow channel at an angle between 1 and 90 degrees.

In various embodiments, the plurality of tines intersects the flow channel at an angle of 45 degrees.

In various embodiments, a height of the sheath fluid channel structure is larger than a height of the flow channel.

In various embodiments, a height of the tines is larger than a height of the flow channel.

In various embodiments, the intersection of each tine in the plurality of tines extends across the flow channel from a first side of the flow channel to a second side of the flow channel.

In various embodiments, a first portion of the sheath fluid channel structure is disposed in a first substrate layer and a second portion of the sheath fluid channel structure is disposed in a second substrate layer.

In various embodiments, a portion of each tine in the plurality of tines for each of the first side channel structure and second side channel structure is disposed above and through a top surface of the flow channel.

In various embodiments, a portion of each tine in the plurality of tines for each of the first side channel structure and second side channel structure is disposed below and through a bottom surface of the flow channel.

In various embodiments, a length of each tine in the plurality of tines of the second side channel structure is shorter than that of each tine in the plurality of tines of the first side channel structure.

In various embodiments, a length of each tine in the plurality of tines of the first side channel structure is longer than that of each tine in the plurality of tines of the second side channel structure.

In various embodiments, a length of each tine in the plurality of tines of the second side channel structure is longer than that of each tine in the plurality of tines of the first side channel structure.

In various embodiments, a length of each tine in the plurality of tines of the first side channel structure is shorter than that of each tine in the plurality of tines of the second side channel structure.

In various embodiments, a width of each tine in the plurality of tines of the second side channel structure is wider than that of each tine in the plurality of tines of the first side channel structure.

In various embodiments, a width of each tine in the plurality of tines of the first side channel structure is narrower than that of each tine in the plurality of tines of the second side channel structure.

In various embodiments, a width of each tine in the plurality of tines of the second side channel structure is narrower than that of each tine in the plurality of tines of the first side channel structure.

In various embodiments, a width of each tine in the plurality of tines of the first side channel structure is wider than that of each tine in the plurality of tines of the second side channel structure.

In various embodiments, the sample fluid channel intersects the flow channel upstream of the intersection of the sheath fluid channel structure and the flow channel.

In various embodiments, a height of the sample fluid channel is equal to a height of the flow channel.

In various embodiments, the flow channel comprises a first geometric focusing region.

In various embodiments, the flow channel comprises a second geometric focusing region.

In various embodiments, the first geometric focusing region comprises a taper.

In various embodiments, the first geometric focusing region comprises a ramp.

In various embodiments, the first geometric focusing region comprises a ramp and a taper.

In various embodiments, the second geometric focusing region comprises a taper.

In various embodiments, the second geometric focusing region comprises a ramp.

In various embodiments, the second geometric focusing region comprises a ramp and a taper.

In various embodiments, the flow channel comprises a detection region.

In various embodiments, a height of the detection region is less than a height of an upstream portion of the flow channel.

In various embodiments, a width of the detection region is less than a width of an upstream portion of the flow channel.

In various embodiments, the detection region is disposed downstream of an intersection of the sheath fluid channel structure and the flow channel.

In various embodiments, the microfluidic channel structure further comprises an expansion region.

In various embodiments, the expansion region is disposed downstream of the detection region.

In various embodiments, a height of the expansion region is larger than a height of an upstream portion of the flow channel.

In various embodiments, a height of the expansion region is larger than a height of the detection region.

In various embodiments, the microfluidic channel structure, further comprises a set of outlet channels.

In various embodiments, the set of outlet channels comprises a single outlet.

In various embodiments, the set of outlet channels comprises three outlets.

In various embodiments, a ratio of widths of the set of outlet channels is 1:2:1.

In various embodiments, the set of outlet channels is disposed downstream of the detection region.

In various embodiments, the set of outlet channels comprise a primary outlet channel and a set of secondary outlet channels.

In various embodiments, the set of secondary outlet channels comprise a set of waste channels.

In various embodiments, the intersection is configured to asymmetrically focus particles in a sample stream flowing through the flow channel.

In various embodiments, the intersection is configured to asymmetrically focus and orient particles in a sample stream flowing through the flow channel.

In various embodiments, the particles are cells.

In various embodiments, the cells are sperm cells.

In various embodiments, the intersection is configured to asymmetrically focus a sample stream.

In various embodiments, the asymmetric focusing comprises flowing a first sheath fluid from a first tine in the plurality of tines of the first side channel structure into the flow channel to direct a sample stream in the flow channel towards a first side wall opposite of the first tine, and flowing a second sheath fluid from a second tine in the plurality of tines of the second side channel structure into the flow channel to direct the sample stream towards a second side wall opposite of the second tine.

In various embodiments, the plurality of tines of the first side channel structure is offset relative to the plurality of tines of the second side channel structure to provide for the asymmetric focusing of the sample stream.

In various embodiments, the asymmetric focusing comprises surrounding the sample stream with sheath fluid from substantially three directions to cause the sample stream to move towards a fourth direction, and subsequently surrounding the sample stream with sheath fluid from substantially three directions to cause the sample stream to move away from the fourth direction.

In various embodiments, the asymmetric focusing comprises repeatedly directing the sample stream towards and away from the fourth direction by repeatedly surrounding the sample stream with sheath fluid from at least three directions.

In various embodiments, the sheath fluid channel structure comprises a herringbone structure configuration.

In various embodiments, each tine in the plurality of tines for the first and second side channel structures comprises a bone of a herringbone structure configuration.

In various embodiments, a width of the main sheath fluid channel is from 50-1000 microns.

In various embodiments, a width of the main sheath fluid channel is 300 microns.

In various embodiments, a height of the main sheath fluid channel is from 100-500 microns.

In various embodiments, a height of the main sheath fluid channel is 400 microns.

In various embodiments, a length of the main sheath fluid channel is from 5000-35000 microns.

In various embodiments, a length of the main sheath fluid channel is 15311 microns.

In various embodiments, a length of the main sheath fluid channel is 24770 microns.

In various embodiments, a width of a tine in the plurality of tines for the first and second side channel structures is from 50-500 microns.

In various embodiments, a width of a tine in the plurality of tines for the first and second side channel structures is 150 microns.

In various embodiments, a width of a tine in the plurality of tines for the first side channel structure is 150 microns and a width of a tine in the plurality of tines for the second side channel structure is 105 microns.

In various embodiments, a width of a tine in the plurality of tines for the first side channel structure and a width of a tine in the plurality of tines for the second side channel structure are not equal.

In various embodiments, a height of a tine in the plurality of tines for the first and second side channel structures is from 100-500 microns.

In various embodiments, a height of a tine in the plurality of tines for the first and second side channel structures is 400 microns.

In various embodiments, a height of a tine in the plurality of tines for the first and second side channel structures is 465 microns.

In various embodiments, a height of a tine in the plurality of tines for the first and second side channel structures is 475 microns.

In various embodiments, a height of a tine in the plurality of tines for the first and second side channel structures is 490 microns.

In various embodiments, a height of a tine in the plurality of tines for the first and second side channel structures comprises 50 microns in a first substrate layer and 350 microns in a second substrate layer.

In various embodiments, a height of a tine in the plurality of tines for the first and second side channel structures comprises 65 microns in a first substrate layer and 400 microns in a second substrate layer.

In various embodiments, a height of a tine in the plurality of tines for the first and second side channel structures comprises 75 microns in a first substrate layer and 400 microns in a second substrate layer.

In various embodiments, a height of a tine in the plurality of tines for the first and second side channel structures comprises 100 microns in a first substrate layer and 300 microns in a second substrate layer.

In various embodiments, a height of a tine in the plurality of tines for the first and second side channel structures comprises 90 microns in a first substrate layer and 400 microns in a second substrate layer.

In various embodiments, a length of a tine in the plurality of tines for the first and second side channel structures is from 1000-15000 microns.

In various embodiments, a length of tines in the plurality of tines for the first side channel structure and a length of tines in the plurality of tines for the second side channel structure are not equal.

In various embodiments, a length of tines in the plurality of tines for the first side channel structure is 1407 microns and a length of tines in the plurality of tines for the second side channel structure is 7460 microns.

In various embodiments, a length of a tine in the plurality of tines for the first and second side channel structures is 7460 microns.

In various embodiments, a distance between a tine in the plurality of tines for the first side channel structure and a tine in the plurality of tines for the second side channel structure at a first side wall of the flow channel is from 600-1500 microns.

In various embodiments, a distance between a tine in the plurality of tines for the first side channel structure and a tine in the plurality of tines for the second side channel structure at a first side wall of the flow channel is 820 microns.

In various embodiments, a width of the flow channel is from 50-500 microns.

In various embodiments, a width of the flow channel is 300 microns.

In various embodiments, a width of the flow channel is 250 microns.

In various embodiments, a width of the flow channel is 300 microns in a first region downstream of the intersection and 250 microns at a second region downstream of the intersection.

In various embodiments, a height of the flow channel is from 100-500 microns.

In various embodiments, a height of the flow channel is 150 microns.

In various embodiments, a height of the flow channel is 67.5 microns.

In various embodiments, a length of the flow channel is from 1001-30000 microns.

In various embodiments, a length of the flow channel is 9200 microns.

In various embodiments, a length of the flow channel is 10912 microns.

In various embodiments, a length of the flow channel is 20112 microns.

In various embodiments, a length of the flow channel is 4912 microns.

In various embodiments, a length of the flow channel is 2319 microns.

In various embodiments, a length of the flow channel is 7231 microns.

In various embodiments, an interrogation region of the flow channel comprises a width of 50-200 microns.

In various embodiments, an interrogation region of the flow channel comprises a width of 135 microns.

In various embodiments, an interrogation region of the flow channel comprises a height of 50-150 microns.

In various embodiments, an interrogation region of the flow channel comprises a height of 67.5 microns.

In various embodiments, an interrogation region of the flow channel comprises a length from 100-5000 microns.

In various embodiments, an interrogation region of the flow channel comprises a length of 2319 microns.

In various embodiments, a sample fluid stream is positioned at a set height within the flow channel.

In various embodiments, a height of the plurality of tines of both the first and second microfluidic channel structure flows a sheath fluid stream into the flow channel to position the sample fluid stream at the set height within the flow channel.

In various embodiments, the set height is at ⅔ of a height of the flow channel, +/−⅛ of the height of the flow channel.

In various embodiments, the set height is at ⅔ of a height of the flow channel.

In various embodiments, the set height is at ⅔ of a height of the flow channel, +/−⅛ of the height of the flow channel.

In various embodiments, the set height is at ¾ of the height of the flow channel.

In various embodiments, the set height is not at a centerline of the flow channel.

In various embodiments, the set height is offset from a centerline of the flow channel.

In various embodiments, the set height is from 18-25 microns from a top surface of the flow channel.

In various embodiments, the set height is at 18 microns from a top surface of the flow channel.

In various embodiments, the set height is at 19 microns from a top surface of the flow channel.

In various embodiments, the set height is at 20 microns from a top surface of the flow channel.

In various embodiments, the set height is at 21 microns from a top surface of the flow channel.

In various embodiments, the set height is at 22 microns from a top surface of the flow channel.

In various embodiments, the set height is at 23 microns from a top surface of the flow channel.

In various embodiments, the set height is at 24 microns from a top surface of the flow channel.

In various embodiments, the set height is at 25 microns from a top surface of the flow channel.

In various embodiments, the set height is at least 18 microns from a top surface of the flow channel and is offset from a centerline of the flow channel.

In another embodiment, what is provided is microfluidic chip comprising: a first substrate layer and a second substrate layer; a sheath fluid channel structure originating upstream at a sheath fluid inlet, the sheath fluid channel structure comprising a first side channel structure and a second side channel structure, wherein an upper portion of the first side channel structure and the second side channel structure are disposed in the first substrate layer, and wherein a lower portion of the first side channel structure and the section side channel structure are disposed in the second substrate layer; a sample fluid channel originating at an upstream sample fluid inlet; a flow channel disposed downstream of the sample fluid inlet and the sheath fluid inlet; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In one embodiment, what is provided is a microfluidic chip comprising the microfluidic channel structure of any of the above embodiments.

In one embodiment, what is provided is a flow cytometry system comprising: an interrogation light source; a detector; and a microfluidic chip, the microfluidic chip comprising: a sheath fluid channel structure originating upstream at a sheath fluid inlet, the sheath fluid channel structure comprising a first side channel structure and a second side channel structure; a sample fluid channel originating at an upstream sample fluid inlet; a flow channel disposed downstream of the sample fluid inlet and the sheath fluid inlet; an interrogation region disposed downstream of the flow channel; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In one embodiment, what is provided is a flow cytometry system comprising the microfluidic chip of any of the above embodiments.

In one embodiment, what is provided is a flow cytometry system comprising a microfluidic chip which comprises the microfluidic channel structure of any of any of the above embodiments.

In one embodiment, what is a method for focusing a sample fluid stream, the method comprising: introducing a sheath fluid as a sheath fluid stream into a sheath fluid channel structure at a sheath fluid inlet; diverting the sheath fluid stream into both a first side channel structure and a second side channel structure of the sheath fluid channel structure; introducing a sample fluid as a sample fluid stream into a sample fluid channel at a sample fluid inlet; flowing the sheath fluid stream into a plurality of tines of the first side channel structure; flowing the sheath fluid stream into a plurality of tines of the second side channel structure; flowing the sample fluid stream into a flow channel disposed downstream of the sample fluid inlet and the sheath fluid inlet; intersecting the sample fluid stream in the flow channel at an intersection with the sheath fluid stream from the plurality of tines of the first side channel structure and from the plurality of tines of the second side channel structure; and asymmetrically focusing the sample fluid stream by the sheath fluid stream at the intersection, wherein the sample fluid stream is focused into a smaller, narrower stream by asymmetric introduction of the sheath fluid stream from the plurality of tines of the first side channel structure and from the plurality of tines of the second side channel structure.

In one embodiment, what is a method for focusing a sample fluid stream, the method comprising: asymmetrically focusing the sample fluid stream by alternately intersecting the sample fluid stream in a flow channel by repeated introduction of a sheath fluid stream.

In one embodiment, what is a method for focusing a sample fluid stream comprising asymmetrically focusing the sample fluid stream in the microfluidic channel structure of any of the above embodiments.

In one embodiment, what is a method for focusing a sample fluid stream comprising asymmetrically focusing the sample fluid stream in a microfluidic chip comprising the microfluidic channel structure of any of any of the embodiments.

In one embodiment, what is provided is a method for focusing a sample stream, the method comprising: positioning the sample stream at a position that is offset from a centerline of a flow channel.

In various embodiments, the method further comprises positioning the sample stream using the microfluidic channel structure of any of the above embodiments.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims . . .

### BRIEF DESCRIPTION OF THE DRAWINGS

In order to facilitate a full understanding of the present invention, reference is now made to the accompanying drawings, in which like elements are referenced with like numerals. These drawings should not be construed as limiting the present invention but are intended to be exemplary and for reference.

FIG. 1 provides a top, plan view of an asymmetrically focusing microfluidic channel structure 100 according to one embodiment.

FIG. 2 provides a top, perspective view of an intersection 600 of an asymmetrically focusing microfluidic channel structure 100 according to one embodiment.

FIG. 3 provides a centerline cross-section view of a cross-channel portion 314 of a tine 312 at an intersection channel 711 of an asymmetrically focusing microfluidic channel structure 100 according to one embodiment.

FIG. 4 provides a top, perspective view of a cross-channel portion 314 of a tine 312 at an intersection channel 711 of an asymmetrically focusing microfluidic channel structure 100 according to one embodiment.

FIG. 5 provides a top, perspective view of a lower or bottom chip layer 101 according to one embodiment.

FIG. 6 provides a detailed perspective view of an intersection 600′ according to one embodiment.

FIG. 7 provides a top, perspective view of an upper or top chip layer 102 according to one embodiment.

FIG. 8 provides a detailed perspective view of an intersection 600″ according to one embodiment.

FIG. 9 provides a top, plan view of a flow simulation of an asymmetrically focusing microfluidic channel structure 100 according to one embodiment.

FIG. 10 provides a detail plan view of a flow simulation of an intersection 600 of an asymmetrically focusing microfluidic channel structure 100 according to one embodiment.

FIG. 11 provides a detail plan view of a flow simulation of an interrogation region 800 of an asymmetrically focusing microfluidic channel structure 100 according to one embodiment.

FIG. 12 provides a detail plan view of a flow simulation of an outlet region 900 of an asymmetrically focusing microfluidic channel structure 100 according to one embodiment.

FIG. 13 provides a side view of a flow simulation of an asymmetrically focusing microfluidic channel structure 100 according to one embodiment.

FIG. 14 provides a detail side view of a flow simulation of an intersection 600 of an asymmetrically focusing microfluidic channel structure 100 according to one embodiment.

FIG. 15 provides a detail side view of a flow simulation of an interrogation region 800 of an asymmetrically focusing microfluidic channel structure 100 according to one embodiment.

FIG. 16 provides a graph of a velocity gradient of an interrogation region of an asymmetrically focusing microfluidic channel structure according to one embodiment.

FIG. 17 provides a graph of sample stream position in an interrogation region of an asymmetrically focusing microfluidic channel structure according to one embodiment.

FIG. 18 provides a graph of a velocity gradient of an interrogation region with a sample stream position at the interrogation region an asymmetrically focusing microfluidic channel structure according to one embodiment.

FIG. 19 provides a representation of the velocity magnitude in opposing sets of tines at an intersection of an asymmetrically focusing microfluidic channel structure according to one embodiment.

FIG. 20 provides a stroboscopic image of cells (bovine sperm cells) at an interrogation region of an asymmetrically focusing microfluidic channel structure according to one embodiment.

FIG. 21 provides a graph of a velocity gradient and sample stream position at an interrogation region of an asymmetrically focusing microfluidic channel structure according to one embodiment.

FIG. 22 provides a microscopy image of particles in a focused sample stream at an interrogation region of an asymmetrically focusing microfluidic channel structure according to one embodiment.

FIG. 23 provides a stroboscopic image of cells (bovine sperm cells) at an interrogation region of an asymmetrically focusing microfluidic channel structure according to one embodiment.

FIG. 24 provides a graph of a velocity gradient and sample stream position at an interrogation region of an asymmetrically focusing microfluidic channel structure according to one embodiment.

FIG. 25 provides a microscopy image of particles in a focused sample stream at an interrogation region of an asymmetrically focusing microfluidic channel structure according to one embodiment.

FIG. 26 provides histogram outputs derived from the interrogation of particles (bovine sperm cells) in a focused sample fluid stream as detected in an interrogation region of an asymmetrically focusing microfluidic channel structure according to one embodiment.

FIG. 27 provides histogram outputs derived from the interrogation of particles (bovine sperm cells) in a focused sample fluid stream as detected in an interrogation region of an asymmetrically focusing microfluidic channel structure according to one embodiment.

FIGS. 28 and 29 provide respective perspective and top plan views of an asymmetrically focusing microfluidic channel structure wherein the length of one set of tines is relatively shorter than the other, corresponding set of tines according to one embodiment.

FIG. 30 provides a graph of a velocity gradient and sample stream position at an interrogation region of the asymmetrically focusing microfluidic channel structure of FIGS. 28 and 29.

FIG. 31 provides a top plan view of an asymmetrically focusing microfluidic channel structure wherein the width of one set of tines is relatively less than the other, corresponding set of tines according to one embodiment.

FIG. 32 provides a top plan view of an asymmetrically focusing microfluidic channel structure wherein the length of one set of tines is relatively shorter than the other, corresponding set of tines according to one embodiment.

FIG. 33 provides a graph of a velocity gradient and sample stream position at an interrogation region of the asymmetrically focusing microfluidic channel structure of FIG. 31.

FIG. 33A provides a detail plan view of a flow simulation of an intersection 3301 of an asymmetrically focusing microfluidic channel structure of FIG. 31.

FIG. 34 provides a graph of a velocity gradient and sample stream position at an interrogation region of the asymmetrically focusing microfluidic channel structure of FIG. 32.

FIG. 35 provides a top plan view of an asymmetrically focusing microfluidic channel structure wherein the length of one set of tines is relatively shorter than the other, corresponding set of tines according to one embodiment.

FIG. 36 provides a graph of a velocity gradient and sample stream position at an interrogation region of the asymmetrically focusing microfluidic channel structure of FIG. 35.

### DETAILED DESCRIPTION

The systems and methods herein will now be described in more detail with reference to exemplary embodiments as shown in the accompanying drawings. While the present invention is described herein with reference to the exemplary embodiments, it should be understood that the systems and methods herein are not limited to such exemplary embodiments. Those possessing ordinary skill in the art and having access to the teachings herein will recognize additional implementations, modifications, and embodiments, as well as other applications for use of the invention, which are fully contemplated herein as within the scope of the systems and methods as disclosed and claimed herein, and with respect to which the systems and methods herein could be of significant utility.

As used herein, “action”, such as in an “action region”, refers to a particular effect that is directed to a thing. Such as, for example, an enrichment, sorting, or effect of a decision that diverts, moves, destroys, or inactivates the thing being acted upon. Without limitation, this may be the laser ablation of a particle in a sample stream, the diversion of a particle from a sample stream, or the trapping or suspension of a particle in a sample stream.

As used herein, “asymmetric” refers to a non-symmetrical or imbalanced structural feature or action. This may be, for example, a physical feature where corresponding elements are positioned at differing or offset locations with respect to a centerline, plane, or axis, or a functional feature, wherein the functional feature is stronger or weaker, more prevalent or less prevalent, or introduced from one side or direction in a greater or lesser degree than from an opposing or corresponding direction. Without limitation, this may be flowing a fluid into a channel from a first and second direction wherein the flow rate is greater or lesser from the first direction than from the second, or wherein the fluid is introduced slightly downstream or upstream in the second direction relative or with respect to the introduction of the fluid into the channel in the first direction.

As used herein, “focusing” refers to the compression, from at least one side, of one fluid stream by another fluid stream in a laminar fluid flow regime in a microfluidic environment especially where particles in or suspended in the first fluid stream are ordered, oriented or aligned with respect to a defined plane or surface, or “focusing” refers to the ordering, orientation, or alignment of particles within a fluid stream in an inertial microfluidic regime by inertial forces within the fluid stream and microfluidic channel. Focusing may be performed by one or more of a geometric channel feature, a channel length or size, or the introduction of one or more sheath or buffer fluids into a flow channel.

As used herein, “intersection”, which may also be referred to as a “junction”, comprises the joining of any two or more channels within the microfluidic channel structure of a microfluidic chip. For example, and without limitation, an intersection may comprise the location where a sheath fluid channel joins a main flow channel, the location where two or more sheath fluid channels join flow channel, the location where a sheath fluid channel and a sample channel join a flow channel, the location where a sheath fluid channel joins a sample channel, or any combination of the aforementioned examples.

As used herein, “interrogation” refers to the process of probing a thing to reveal one or more characteristics of a thing, such as the identification of a characteristic of a particle. This may be, for example, the identification of a characteristic of a particle by exposing the particle to a light source, such as a laser beam, and detecting one or more light emissions from the particle, such as forward scatter, side scatter, or a Stoke-shifted emission.

As used herein, “laminar flow” refers to a non-mixing flow regime in a microfluidic environment where one or more flow streams flow through a flow channel in a non-mixing manner. Laminar flow may be defined as a flow having a particular stream or particle Reynold's number.

As used herein, “particle” refers to any thing which may be present in a flow stream. For example, a particle may be a polystyrene bead, a magnetic bead, a protein, cellular debris, small inorganic matter, or a cell, such as a sperm cell.

As used herein, “sample fluid” refers to a fluid comprising an analyte to be measured. A sample fluid may be, for example, a fluid mixture comprising a buffer fluid media and a plurality of polystyrene beads, or may be a fluid mixture of sperm cells in seminal plasma, or may be a fluid mixture of sperm cells, with or without seminal plasma, in an other media.

As used herein, “sheath fluid” or “buffer fluid” refers to a fluid which provides sheath flow and/or hydrodynamic focusing of a sample fluid in a microfluidic environment in laminar flow conditions.

As used herein, “structure” is an element or collection of elements of a physical thing. For example, a sheath fluid structure may comprise one or more sheath fluid channels and all elements or components of the sheath fluid channels. For example, a microfluidic channel structure may comprise all elements therein, such as sample fluid channels, flow channels, sheath fluid channels, and outlet channels. A structure may comprise one or more other structures.

As used herein, “tine” refers to a smaller branching element of a channel and may also be referred to herein as a “bone” or “branch” of a channel or microfluidic channel structure. For example, a “herringbone” structure may comprise one or more sets of tines or “bones”.

As used herein, “set” refers to a collection or grouping of one or more things, especially similar, associated, or related things, and which does not refer to a null set or null collection unless explicitly specified.

While structures, channels, and other features herein may be referred to as a “first” or a “second” structure or feature, this is for reference only, and the structures or features may also be considered to be a “top” and a “bottom”, a “left side” and a “right side”, “corresponding”, or “opposing” structures unless a particular structural or spatial arrangement is explicitly indicated as necessary for function or operation.

Generally, a microfluidic system having a microfluidic chip with a microchannel is disclosed. In one example, the microfluidic system includes at least one detection means, such as a detection site or any other detection means. The microfluidic chip may be used with the as least one detection means. The microchannel is configured to receive fluid having particles. The geometry of sections of the microchannel, the flow of a buffer or sheath fluid, and a pressure and flow rate of the fluid through the microchannel optimize focusing and orientation of the particles within the microchannel, orienting the particles.

The present invention relates to a microfluidic chip with an interrogation apparatus that detects and interrogates objects in a sample fluid mixture, and a focused energy apparatus that performs an action on the objects or a region around the objects. In one embodiment, the interrogation apparatus interrogates the objects to identify the objects and to determine whether the objects should be targeted by the focused energy apparatus. In one embodiment, the targeted objects are unwanted targeted objects.

In one embodiment, the focused energy apparatus is a discrimination apparatus that discriminates between targeted and non-targeted objects by damaging, killing, altering, disabling, or destroying the targeted objects. The present invention is conducted in a flowing, continuous fluid stream within the microfluidic network, where objects are subject to hydrodynamic focusing, positioning, and orientation, and non-targeted objects are allowed to flow through the microfluidic chip undisturbed, and targeted objects may be acted upon, including photodamaged, killed, altered, disabled, or destroyed, by a focused energy apparatus.

The various embodiments of the present disclosure provide for the selection of objects in a fluid mixture, such as, for example: selecting viable or motile sperm from non-viable or non-motile sperm; selecting sperm by gender, and other sex selection variations; selecting stems cells from cells in a population; selecting one or more labeled cells from un-labeled cells distinguishing desirable/undesirable traits; selecting cells for desirable characteristics; selecting genes in nuclear DNA in cells, according to a specified characteristic; selecting cells based on surface markers; selecting cells based on membrane integrity (viability), potential or predicted reproductive status (fertility), ability to survive freezing, etc.; selecting cells from contaminants or debris; selecting healthy cells from damaged cells (i.e., cancerous cells) (as in bone marrow extractions); red blood cells from white blood cells and platelets in a plasma mixture; and selecting any cells from any other cellular objects, into corresponding fractions; selecting damaged cells, or contaminants or debris, or any other biological materials that are desired to discriminated. The objects may be cells or beads treated or coated with linker molecules or embedded with a fluorescent or luminescent label molecule(s). The objects may have a variety of physical or chemical attributes, such as size, shape, materials, texture, etc.

In one embodiment, a heterogeneous population of objects may be measured, with each object being examined for different quantities or regimes in similar quantities (e.g., multiplexed measurements), or the objects may be examined and distinguished based on a label (e.g., fluorescent), image (due to size, shape, different absorption, scattering, fluorescence, luminescence characteristics, fluorescence or luminescence emission profiles, fluorescent or luminescent decay lifetime), and/or particle position, etc.

In addition, the subject matter of the present disclosure is also suitable for other medical applications as well. For example, the various laminar flows discussed below may be utilized as part of a kidney dialysis process, in which whole blood is cleansed of waste products and returned to the patient. Further, the various embodiments of the present disclosure may have further applicability to other biological or medical areas, such as for selection of cells, viruses, bacteria, cellular organelles or subparts, globular structures, colloidal suspensions, lipids and lipid globules, gels, immiscible particles, blastomeres, aggregations of cells, microorganisms, and other biological materials. For example, the object selection in accordance with the present disclosure may include cell “washing”, in which contaminants (such as bacteria) are removed from cellular suspensions, which may be particularly useful in medical and food industry applications. Further, the present invention has the applicability to select non-motile cellular objects from motile cellular objects.

The subject matter of the present disclosure may also be utilized to transfer a species from one solution to another solution where separation by filtering or centrifugation is not practical or desirable. In addition to the applications discussed above, additional applications include selecting colloids of a given size from colloids of other sizes (for research or commercial applications), and washing particles such as cells, egg cells, etc. (effectively replacing the medium in which they are contained and removing contaminants), or washing particles such as nanotubes from a solution of salts and surfactants with a different salt concentration or without surfactants, for example.

The action of selecting species may rely on a number of physical properties of the objects or objects including self-motility, self-diffusivity, free-fall velocity, or action under an external force, such as an actuator, an electromagnetic field, or a holographic optical trap. The properties which may be selected include, for example, cell motility, cell viability, object size, object mass, object density, the tendency of objects to attract or repel one another or other objects in the flow, object charge, object surface chemistry, and the tendency of certain other objects (i.e., molecules) to adhere to the object.

In one embodiment, the optimized focusing and orientation of the particles by the microfluidic chip of the present disclosure provides for detection by at least one detection means, and the detection comprises a detection of a difference in DNA content in the particles. The difference in DNA content may comprise one or more of: (1) approximately a 4% difference in DNA content; or (2) the presence or the absence of an X/Y chromosome, for example. For example, in some examples, the at least one detection means may include one or more of the following embodiments: (1) detecting cells as being live or dead, and acting on a subset of the live cells based upon a further classification; (2) detecting the presence of an X or Y chromosome in cells, identifying cells comprising a Y chromosome, and acting on the Y chromosome bearing cells; (3) detecting the presence of an X or Y chromosome in cells, identifying cells not comprising an X chromosome, and acting on the not-X cells; (4) detecting the presence of an X or Y chromosome in cells, identifying cells comprising a X chromosome, and acting on the X chromosome bearing cells; and/or (5) detecting the presence of an X or Y chromosome in cells, identifying cells not comprising a Y chromosome, and acting on the not-Y cells. Further, although the above-described example is specific to X/Y chromosome detection, it will be understood that other DNA segments may be used in the systems and methods of the present invention and still fall within the scope of the present disclosure. For example, other DNA segments could be labeled and the same methods used to de-activate sperm cells with undesired traits.

While discussion herein may focus on the identification and selection of viable or motile sperm from non-viable or non-motile sperm, or selecting sperm by gender and other sex selection variations, or selecting one or more labeled cells from un-labeled cells distinguishing desirable/undesirable traits, etc., the apparatus, methods, and systems of the present invention may be extended to other types of particulate, biological or cellular matter, which are capable of being interrogated by fluorescence techniques within a fluid flow, or which are capable of being manipulated between different fluid flows into one or more outputs.

The various embodiments of the microfluidic chip, as described herein, utilize one or more flow channels, having a set of substantially laminar flows, allowing one or more objects to be interrogated for identification by an interrogation apparatus, and to be acted upon, such as by an actuation or by a focused energy apparatus, with the objects exiting the microfluidic chip via one or more outputs. In one embodiment, the objects not acted upon are undisturbed, and the action diverts, traps, photodamages, alters, disables, kills, or destroys targeted objects.

The various embodiments of the present invention thereby provide a selection of objects on a continuous basis, such as, within a continuous, closed system without the potential damage and contamination of prior art methods, particularly as provided in sperm separation. The continuous process of the present invention also provides significant time savings in selecting and discriminating objects.

The sheath or buffer fluids are well known in the art of microfluidics, and in one embodiment, may contain nutrients well known in the art to maintain the viability of the objects (i.e., sperm cells) in the fluid mixture. Commercially available Tris, as sold by Chata Biosystems, is one example, and the sheath or buffer fluid 163 may be formulated to include the following: Water—0.9712 L; Tris—23.88 gg; citric acid monohydrate—11.63 g; D-fructose—8.55 g. The pH is adjusted to 6.80±0.05 with hydrochloric acid, and osmolarity is adjusted, if necessary, to 270-276 mOsm with fructose high purity. The mixture is filtered using a 0.22-micron filter.

The microfluidic chip may have one or more structural layers in which the micro-channels are disposed. The channels may be disposed in one or more layers or in-between layers. Any suitable bonding process known by one of ordinary skill in the art would provide for a microfluidic chip, channel, or device as described herein. For example, in injection molding, instead of forming two layers, two molds could be made and joined together, such that an injection is made into the cavity in order to obtain the chip of the present invention. Layers may also be separately etched in glass or another material, such as by chemical, optical, or cutting processes, and joined or bonded together in a proper alignment to form a complete microfluidic chip.

However, one of ordinary skill in the art would know that more or fewer structural layers with functional sides, and with or without “blank” layers, may be used, and the channels may be disposed in any of the structural layers, or in different structural layers, and in any arrangement, with access to those channels through a top “blank” layer, as long as the object of the present invention is achieved.

In conventional flow cytometry systems, such as droplet-in-air flow cytometry systems, since objects or cells, especially with asymmetric shapes, tend to orient as they flow close to a solid surface, the function and improvement of object or cell orientation relies on complex nozzle designs, such as orienting baffles or an offset structure inside the nozzle. To avoid the complex design of nozzle-based flow cytometry systems, and their high fabrication cost, the microfluidic chip and channel design described herein focuses, positions, and orients the particles in order to optimize the analytical capability of a flow cytometry system or apparatus which interrogates and acts upon the particles. Thus, in one embodiment, the particles (i.e., cells which may have or comprise asymmetric or non-spherical shapes) are aligned into a restricted core volume, which may be referred to as a core stream having a width of 2-100 microns and a height of 2-100 microns, in a channel and maintained in a similar and desired orientation as the particles pass through an interrogation region. As a result, more uniform scattering and detection signals is obtained, thus, helping to increase the system's sensitivity and stability.

The design of the microfluidic chip and channel structure and the flow cytometry system described herein provides for the core sample stream to orient flat-shaped particles, position the particles in a channel in a physical arrangement approaching uniformity, all of which improves the downstream precision action by a device, such as a focused energy apparatus or jet-sorting fluid actuator, performing an action on the particles.

To realize the exemplary three-dimensional hydrodynamic focusing methods described herein, both sample fluid and sheath or buffer fluids are required to be precisely delivered so that a constant flow can be streamed through the microfluidic chip. After being compressed by the sheath or buffer fluid flows, the particles have been accelerated and the average spacing between the particles in the sample core stream is also stretched significantly therefrom. The ratio of the total sheath or buffer fluid flow rate and the sample flow rate can be adjusted between 100:1 and 1000:1. Preferably, the ratio of 200-400:1 is used in the microfluidic chip and channel structure described herein. The overall fluid flow rate in the microfluidic chip and channel structure is about 2-4 ml/min, 2.5 ml/min, 3 ml/min, 4-6 ml/min, or 1-2 ml/min so long as laminar flow (non-turbulent flow) of the sample stream and the sheath fluid stream is maintained. The introduced sheath or buffer fluid flows are constant and pulse-free to ensure a stable traveling speed of the particles during interrogation and signal detection, and between the detection/interrogation position in the interrogation region and the position of the action on the particles downstream of the interrogation. This facilitates an accurate signal reading and action on a target particle by, for example, a focused energy apparatus. With the precise control of fluid flow through the main channel (a flow channel), the overall flow rate variation is less than 1% of the set flow rate, and the traveling speed of target particles for potential action varies less than 1% from the position where interrogation and detection of particles takes place, to the position where the particles are acted upon.

One of the challenging issues in the detection of flat-shaped particles (i.e., sperm cells) is to constrain the particles in a uniform orientation when passing through, for example, an interrogation beam of electromagnetic radiation emitted from a laser at an interrogation location within an interrogation region. Thus, an approximately uniform positioning of particles and a corresponding orientation of particles in the flow channel helps to increase the sensitivity of the system. With the asymmetric hydrodynamic focusing strategy described herein, the position of particles along the flow channel can be manipulated in a controlled way. Thus, by adjusting the ratio between the sheath or buffer fluid flows from asymmetrical positioned or configured side channel structures through either they physical configuration of the side channel structures or the flow rate of the sheath or buffer fluid therethrough, a position of the focused sample stream offset from the center of the channel by about 5-40 microns (based on a channel cross-section of 150-micron width, and 100-micron heights, for example), is preferred for the detection of flat-shaped particles. Generally, an offset of 0-100 microns bias position of the particles can be achieved as either a fixed (e.g., from a physical channel configuration) or adjustable (e.g., from varying the flow rates) offset.

In one embodiment, pancake-shaped, or oar-shaped sperm cells are taken as an example of the particles. Because of their pancake-type or flattened teardrop-shaped heads, the sperm cells will re-orient themselves in a predetermined direction as they undergo asymmetric focusing—i.e., with their flat surfaces perpendicular to the direction of an interrogation light source. Thus, the sperm cells develop a preference on their body orientation while passing through the asymmetric hydrodynamic focusing process. Specifically, the sperm cells tend to be more stable with their flat bodies perpendicular to the direction of the compression, with additional orientation achieved based on shear forces associated with a proximity to a surface, such as a top surface of a flow channel. Hence, with the design or control of the flow of sheath or buffer fluids, such as asymmetrically flowing the fluids, the sperm cells which start with random orientation, now achieve uniform orientation. Thus, the sperm cells are not only disposed in a restricted core volume at the center of the flow channel, but they also achieve a uniform orientation.

The asymmetric focusing improves the sperm cells' orientation and the capability to differentiate the DNA content of X- and Y-sperm chromosomes (and thereby distinguish between X and Y sperm).

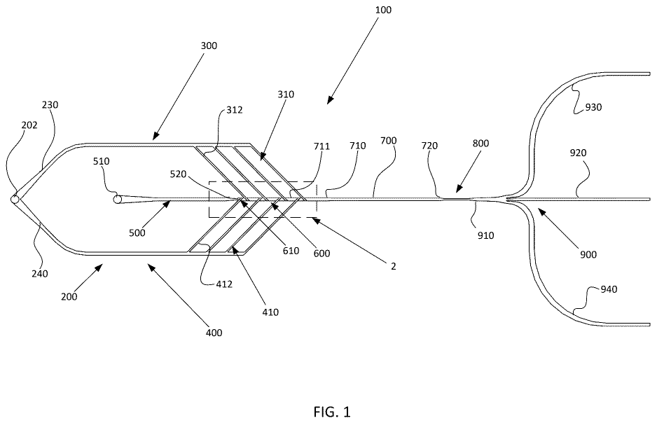

Now, with respect to FIG. 1, what is provided is a top plan view of a microfluidic channel structure 100 adapted or configured to hydrodynamically focus, orient, and/or align particles suspended in a sample fluid as the sample fluid flows from an inlet, through a flow channel, and out from one or more outlets. The microfluidic channel structure 100 comprises a sheath fluid channel structure 200, a sample fluid channel 500, a flow channel 700, an intersection 600, an interrogation region 800, which may also be referred to and/or comprise an action or inspection region, and an outlet region 900.

In operation, a sheath or buffer fluid, which is a fluid suitable for hydrodynamic focusing in a microfluidic environment, is introduced into the sheath fluid channel structure 200 through the sheath fluid inlet 202. The sheath fluid is diverted, flowed, or directed into a first 230 and a second 240 sheath fluid channel. The first 230 and second 240 sheath fluid channels divert or direct the sheath fluid to flow into a respective first side channel structure 300 and a second side channel structure 400. Each of the first side channel structure 300 and second side channel structure 400 comprise a plurality of tines, respectively the first plurality of tines 310 and the second plurality of tines 410. Each plurality of tines comprises a set of one or more tines, such as the set of four (4) tines shown in FIG. 1. For example, the first plurality of tines 310 includes at least the tine 312, and the second plurality of tines 410 includes at least the tine 412.

The sheath or buffer fluid flows through the first plurality of tines 310 and the second plurality of tines 410 into the intersection 600 to asymmetrically focus a sample fluid. The sample fluid is flowed into the microfluidic channel structure 100 via the sample fluid inlet 510 and flows through the sample fluid channel 500 to the intersection. The sample fluid channel 500 may comprise a geometric feature 520, which comprises a ramp and/or a taper to provide one or both of a reduction in channel size horizontally or vertically, and which is used to position the sample fluid channel 500 with respect to the intersection 600 and flow channel 700 and to condition the flow characteristics of a sample fluid within the channel, such as to increase its velocity.

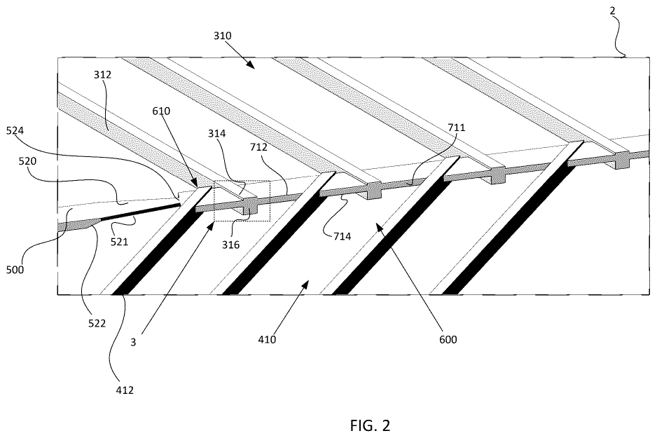

In the intersection 600, shown in greater detail as detail 2 in FIG. 2, the sheath or buffer fluid from the first plurality of tines 310 and the second plurality of tines 410 flows into the intersection channel 711 of the flow channel 700. The intersection channel 711 is a portion or element of the flow channel 700 in the intersection 600 to focus and flow laminarly with the sample fluid from the sample fluid channel 500. At the first intersection 610 of the intersection 600, sheath or buffer fluid flowing from the tines 312 and 412 flows into the intersection channel 711 of the flow channel 700 after the introduction of the sample fluid flowing into the intersection channel 711 from the sample fluid channel 500.

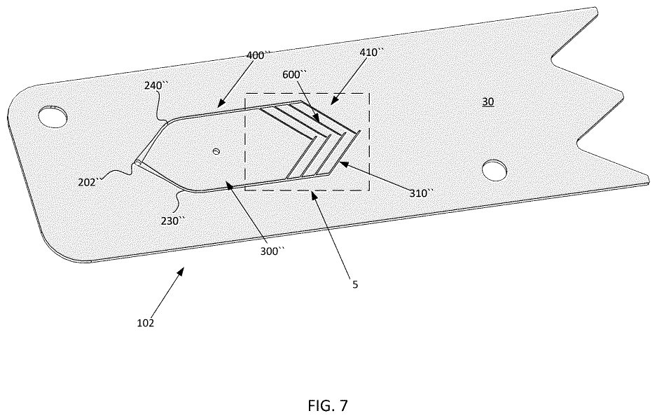

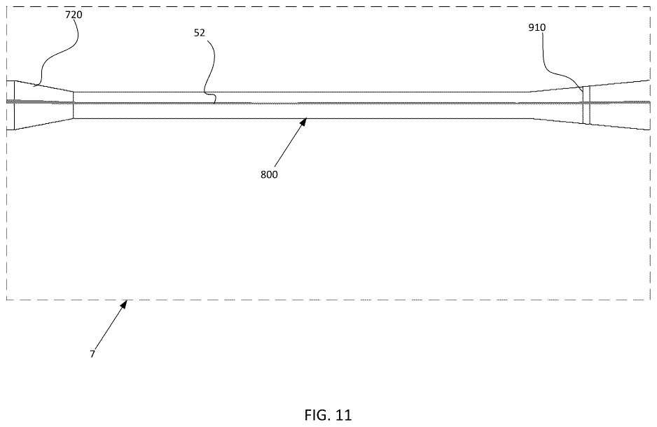

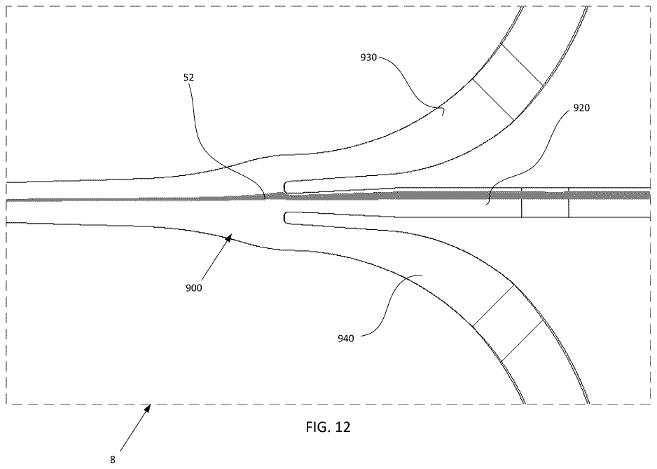

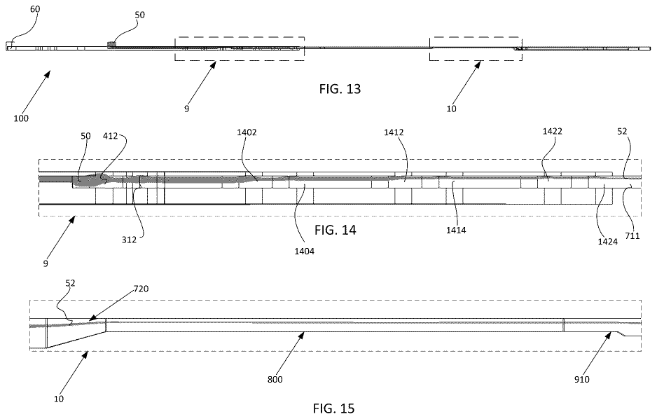

At the first intersection 610, and at any number of subsequent intersections or junctions where tines introduce sheath fluid into the flow channel in the intersection, the volume or rate of flow of sheath or buffer fluid from the sheath fluid channel structure 200 into the intersection via the tines (e.g., tines 312 and 412) governs or dictates the manner of asymmetric hydrodynamic focusing of the sample fluid within the intersection. As is shown in greater detail in FIGS. 9-15, the sheath or buffer fluid focuses the sample fluid substantially in a first direction and then in a second direction away from the first direction. The volume of flow from each tine will control the amount the sample fluid is focused in any one direction, and biasing the flow of one set of tines (e.g., first set of tines 310) to be greater or less than another set of tines (e.g., second set of tines 410) can be used to position the sample fluid stream within the flow channel 700 and can be used to control the amount and rate of focusing of the sample fluid stream. For example, the rate or volume of flow from the first set of tines 310 can be controlled, through channel size (e.g., width and height) or channel length, or by controlling the flow rate via a flow controller or pump, to be greater or less than the rate or volume of flow of the second set of tines 410.

In one exemplary embodiment, the rate or volume of flow from a first set of tines may be reduced or lesser than the rate or volume of flow of a second set of tines by having the cross-sectional area of the first set of tines be less than the cross-sectional area of each tine in the first set of tines be less than each tine (e.g., corresponding or opposing tines) in the second set of tines. In another exemplary embodiment, the rate or volume of flow from a first set of tines may be reduced or lesser than the rate or volume of flow of a second set of tines by having the length of each tine in the first set of tines be longer than the length of each tine (e.g., corresponding or opposing tines) in the second set of tines. In either embodiment, the focusing of the sample fluid in the intersection would be biased towards the first set of tines and away from the second set of tines.

At the first intersection 610, the sheath or buffer fluid from the tine 412 intersects the sample fluid first. The sheath or buffer fluid from the tine 412 would surround the sample fluid on three sides (e.g., a first vertical side (which may be a left or a right side), a top, and a bottom) and compress, direct, or focus the sample fluid towards a fourth side (e.g., a second vertical side opposite the first vertical side in the intersection channel 711). The sheath or buffer fluid from the tine 312 would then surround the sample fluid from three sides, one of which being the fourth side, and compress, direct, or focus the sample fluid towards the direction of the tine 412 (e.g., towards the first vertical side).

This alternating focusing is asymmetric focusing. Asymmetric focusing is the focusing of the sample fluid primarily from or towards a first direction and then subsequently away from the first direction. This may be achieved through, as shown in FIG. 1, alternate introduction of sheath or buffer fluid (e.g., from tines 412 and then 312), and/or from the varying volume or rate of flow of the sheath or buffer fluid from the tines. The asymmetric focusing is relative to any position along a center line of the flow channel 700, especially with respect to a flow direction or direction of flow of the sample fluid and/or sheath or buffer fluid. The asymmetric focusing may be repeated any number of times to achieve a desired level or quality of focusing of the sample fluid stream within the flow channel 700. For example, as shown in FIG. 1, the asymmetric focusing is repeated four (4) times, but it has been found by the inventors to be repeatable from 2-8 times to achieve varying levels or qualities of focusing based upon the type of sample fluid and sheath or buffer fluid used and the flow rates, characteristics, or conditions of the fluids (e.g., pressure, velocity, viscosity, concentration).

Downstream in a flow direction of the intersection 600, and after the asymmetric focusing of a sample fluid by the introduction of a sheath or buffer fluid from the sets of tines 310 and 410, the sample and sheath or buffer fluids flow laminarly down the flow channel 700 where one or more geometric restrictions, or changes in channel dimension, may further focus or condition the sample stream prior to the interrogation of particles in the sample stream in the interrogation region 800. For example, the restrictions 710 and 720 may be lateral tapers which taper from one or both sides to reduce the channel width cross-section horizontally, and/or may be vertical ramps with reduce the channel height cross-section vertically. As shown in FIG. 1, the first restriction 710 is a geometric channel feature which reduces the channel width horizontally from a first width to a smaller, second width, and the second restriction 720 is a geometric channel feature which reduces the channel width horizontally and height vertically from a first width and a first height to a smaller second width and smaller second height. In another embodiment, the second restriction 720 comprises only a reduction in width and not height. In another embodiment, the flow channel comprises only the first restriction 710. In another embodiment, the flow channel comprises only the second restriction 720. In another embodiment, the flow channel does not comprise either the first restriction 710 or the second restriction 720.

Further downstream is the interrogation region 800. In a preferred embodiment, the width of the microfluidic channel at the interrogation region 800 is less than that of the flow channel 700, but the width and height may both be less than that of the flow channel 700, or may be the same depending on the type, nature, and characteristics of the particles and sample fluid being interrogated. The purpose of the interrogation region 800 is to optimally position and condition the flow stream, comprising the sample fluid stream and the sheath or buffer fluid stream, for interrogation by an interrogation means. In one embodiment, the interrogation means is an emitter of light and a detector. The emitter of light may be an emitter of electromagnetic radiation, such as a laser, which may be a continuous wave laser or a pulsed laser, or the emitter may be another light source such as a light emitting diode or an arc lamp. The detector may be any suitable detector such as an avalanche photodiode or a photomultiplier tube. The emitter and detector are used to interrogate particles within or suspended in the sample fluid steam, such as dye-stained sperm cells. However, other particle types and other interrogation, tagging, and identification methods are contemplated herein.

The interrogation region 800 may further comprise an action region. The action region may be a portion downstream of an interrogation and detection of a particle wherein a particle is acted upon. For example, a particle may be acted upon by the or another electromagnetic radiation emission source to deactivate, slice, kill, or ablate the particle. In another embodiment, an actuator is used to generate a pulse or jet of fluid to divert a particle from the sample fluid stream into the sheath or buffer fluid stream, or into another waste fluid stream. In another embodiment, a series of light sources or waveguides may be used as optical traps or tweezers to divert a particle from the sample fluid stream. In whichever form the interrogation and action take place within, or after, the interrogation region, the asymmetric focusing in the intersection 600 will have properly conditioned, such as by orienting, aligning, and or spacing, such as in a substantially single file line, the particles in the sample fluid for interrogation.

The outlet region 900 comprises an expansion region 910 and first 920, second 930, and third 940 outlet channels. The outlet channels 910, 920, and 930 each comprise an outlet which provides for fluids and/or particles to exit the microfluidic channel structure 100. Downstream of the interrogation region 800, the outlet region 900 provides for a reduction in pressure via a widening of the channel height and/or width at the expansion region 910. In one embodiment, the sample fluid substantially exits the microfluidic channel structure 100 via the outlet 920, and sheath or buffer fluid exits though the outlets 930 and 940. In another embodiment, only desired particles in the sample fluid exit through the outlet 920, while waste components and sheath or buffer fluid exit through the outlets 930 and 940.

With reference now to FIG. 2, a top perspective view of the region 2 including the intersection 600 of the microfluidic channel structure 100 is provided. In the region 2, the end of the sample fluid channel 500 is shown as it leads into and joins the intersection channel 711 of the flow channel 700. A ramp 522 and a taper 521 in the reduction region 520 reduce the width and height of the sample fluid channel 500 to increase the velocity or flow rate of a sample fluid stream flowing therein. At the sample fluid outlet or introduction point 524, the height of the sample fluid channel 500 is the same as, and optionally may be less than, a height of the intersection channel 711 of the flow channel 700. The height of the intersection channel 711 is constant from the introduction point 524 and through the intersection 600, exclusive of the tines.

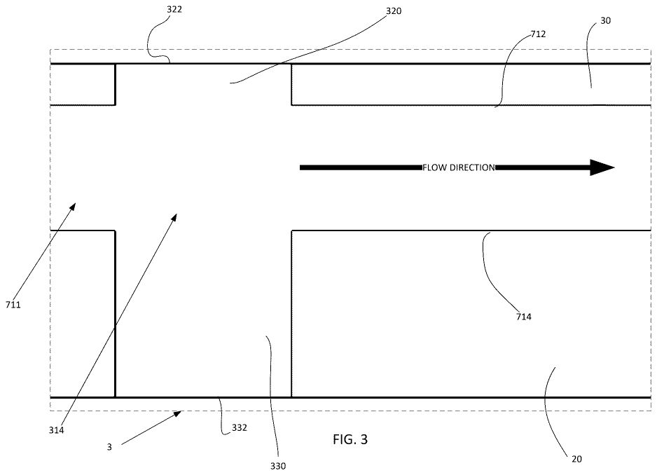

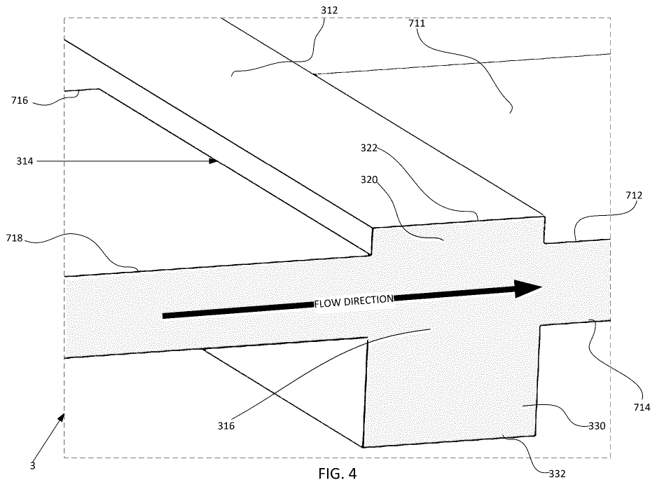

At the first intersection 610, the tine 412 from the second set of tines 410 and the tine 312 from the first set of tines 310 join the intersection channel 711. As is shown in FIG. 2, and in more detail in region 3 of FIGS. 3 and 4, each tine (e.g., tines 312 and 412) extends fully across the intersection channel 711 from one side to an opposite side. In addition, a portion of the height of each tine extends above and below the top 712 and bottom 714 surfaces of the intersection channel 711. As shown in FIG. 2, a cross-channel portion 314 of the tine 312 extends fully across the intersection channel 711 to the terminating surface 316, which is on an opposite or opposing side of the intersection channel 711 from where the tine 312 joined the intersection channel. Each intersection or junction, such as the first intersection 610, in the intersection 600 may be identically or substantially similarly configured, or may be varied in configuration to provide for a desired focusing or flow condition of the sheath fluid and sample fluid at the termination or end of the intersection 600. In an exemplary embodiment, and as shown in FIG. 2, each intersection is identical in its geometric configuration to each other intersection. The intersection 600 may be considered a focusing region or may be a component of a focusing region which further comprises other geometric channel features (e.g., the restrictions 710 and 720 as shown in FIG. 1). In one embodiment, a focusing region comprises the intersection 600. In another embodiment, the focusing region comprises the intersection 600 and at least one of the restrictions 710 and

With reference now to FIG. 3, a side view of the region 3 including the cross-channel portion 314 of the tine 312 of the microfluidic channel structure 100 is provided. As shown in region 3, the portion of the intersection 600 is at a centerline of the intersection channel 711 and further shows a first or lower substrate 20 and a second or upper substrate 30. In this embodiment, the upper surface or top 712 of the intersection channel 711 is defined by a bottom of the upper substrate 30, and the bottom 714 of the intersection channel 711 is disposed within the lower substrate 20. An upper portion 320 having a top 322 and a lower portion 330 having a bottom 332 of the tine 312 (shown here as the cross-channel portion 314) extend above and below the top 712 and bottom 714 respectively of the intersection channel 711. The height of the tine 312, including at the cross-channel portion 314, is greater than the height of the intersection channel 711. The cross-channel portion 314 of the tine 312 extends fully across the intersection channel 711. As shown in region 3 of FIG. 2, a flow of a sheath or buffer fluid would be towards the viewing plane and orthogonal to the flow direction indicated. The sheath or buffer fluid would surround, at least on three sides, a sample flow stream and would join the flow in the intersection channel 711 and continue to flow downstream in the indicated flow direction. Each tine in the sets of tines 310 and 410 would be similarly configured, with opposing, corresponding tines being introduced in opposite directions and slightly offset, in flow direction, from one another to asymmetrically focus the sample fluid stream.

With reference now to FIG. 4, a side perspective view of the region 3 including the terminating surface 316 of the tine 312 of the microfluidic channel structure 100 is provided. The cross-channel portion 314 of the tine 312 extends across the intersection channel 711 from a first side 716 to a second side 714, comprises an upper portion 320 having a top 322 which extends above the top 712 of the intersection channel 711, and comprises a bottom portion 330 having a bottom 332 which extends below the bottom 714 of the intersection channel 711. Sheath or buffer fluid flowing through the tine 312 would flow into the intersection channel 711 starting at the first side 716 where the tine 312 intersects the intersection channel 711, but would continue to flow through the cross-sectional area or volume of the cross-channel portion 314 until the sheath or buffer fluid met the terminating surface 316. In this manner, the sheath or buffer fluid would surround a sample fluid on at least three sides, or primarily on three sides, but may also partially envelop the sample fluid on a fourth side based upon the fluid dynamics of the interaction of the sheath or buffer fluid with the terminating surface 316. In any embodiment, laminar flow is maintained between the sheath or buffer fluid and the sample fluid in the area shown in region 3. Moreover, similar interactions between a sheath or buffer fluid flowing into the intersection region 711 from a tine would be found at any intersection of a tine with the intersection region 711.

TABLE 1, below, provides exemplary dimensions for varying elements or 5 structures of the microfluidic channel structure 100. Ranges with exemplary values are provided.

TABLE 1

| | Dimension | | | |
|---|---|---|---|---|
| | Width | Height | | |
| | (μm) | (μm) | Length (μm) | Angle (°) |
| Sheath fluid channels | 50-1000 | 100-500 (350; | 5000-40000 | N/A |
| (bottom) | (300) | 400) | (15311; 15312; | |
| | | | 24770; 32720) | |
| First set of tines (bottom) | 50-500 (105; | 100-500 (350; | 1000-10000 | N/A |
| | 150) | 400) | (1407; 7460) | |
| Tine angle | N/A | N/A | N/A | 1-90 (45) |
| Inter-tine distance | N/A | N/A | 600-1500 (820) | N/A |
| Second set of tines | 50-500 (105; | 100-500 (350; | 1000-15000 | N/A |
| (bottom) | 150) | 400) | (1407; 7460) | |
| Intersection channel | 100-500 | 50-500 (67.5; | 6500-15000 | N/A |
| | (300) | 75; 150) | (7250; 9200) | |
| Flow Channel | 50-500 (250) | 50-500 (67.5; | 1000-15000 | N/A |
| | | 75; 150) | (4912; 10912) | |
| Interrogation Region | 50-200 (135) | 50-150 (67.5; | 100-5000 | N/A |
| | | 75) | (2319) | |
| Sheath fluid channels | 50-1000 | 10-200 (50; | 5000-35000 | N/A |
| (upper) | (300) | 65; 100) | (16045) | |
| Second set of tines (upper) | 50-500 (105; | 50-500 (50; | 1000-15000 | N/A |
| | 150) | 65; 75; 90; | (1407; 7460) | |
| | | 100) | | |
| First set of tines (upper) | 50-500 (105; | 50-500 (50; | 1000-10000 | N/A |
| | 150) | 65; 75; 90; | (1407; 7460) | |
| | | 100) | | |

With reference now to FIGS. 5-8, perspective views of a first or bottom chip layer 101 and a second or upper chip layer 102 comprising a microfluidic channel structure 100′ which may be joined or bonded together to form a microfluidic chip are provided. The microfluidic channel structure 100′ is substantially similar to the structure 100 described herein, and any structures identified by a reference number and a prime or double-prime comprise identical features and functionality to those described with respect to the microfluidic channel structure 100. The microfluidic channel 100′ shown in the upper chip layer 102 is shown as reversed in orientation from the channel 100′ shown in the lower chip layer 101 as the upper chip layer 102 would be rotated 180 degrees along its longitudinal axis to be disposed above the lower chip layer 101. One or more registration marks may be used to align the upper 102 and lower 101 chip layers.

In an exemplary embodiment, the upper chip layer 102 comprises the upper substrate 30 and bottom chip layer 101 comprises the lower substrate 20, and each of the substrates comprise glass or PDMS, but in other embodiments may also comprise any other material suitable for use as a substrate for a microfluidic chip. However, when used with a laser, such as a laser operating at UV or near-UV wavelengths, a glass, such as, for example, a boro-silicate glass, would be preferable. Most of the microfluidic channel structure 100′ is formed, such as by wet etching, lithography, electron discharge machining, or other known technique, in the bottom substrate 20. A portion of the microfluidic channel structure 101 is also formed in the upper substrate 30. The bottom substrate 20 comprises a portion of the sheath fluid inlet 202′ the first 230′ and second 240′ sheath fluid channels, and a portion of the sheath fluid channel structures 300′ and 400′ including the pluralities of tines 310′ and 410′. The upper substrate 30 comprises a portion of the sheath fluid inlet 202″ the first 230″ and second 240″ sheath fluid channels, and a portion of the sheath fluid channel structures 300″ and 400″ including the pluralities of tines 310″ and 410″. The sample fluid channel 500′ including the sample fluid inlet 510′ is formed in the bottom substrate 20.

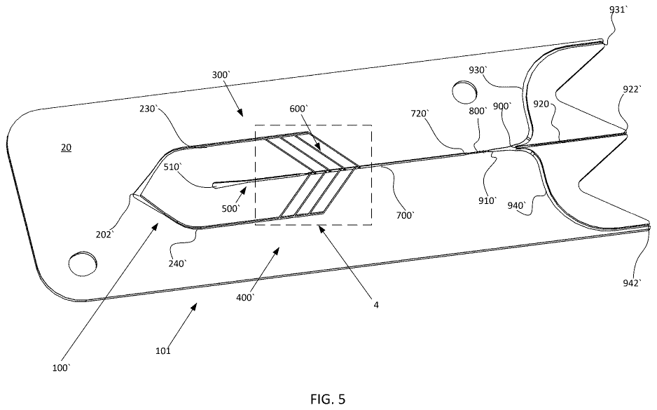

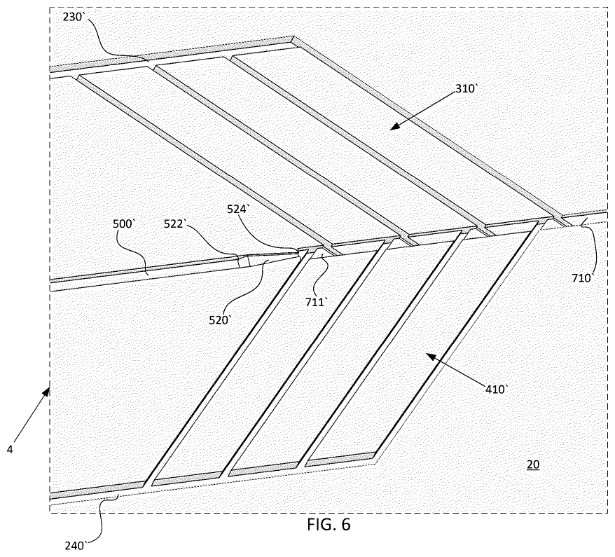

The intersection, shown in regions 4 and 5, and in greater detail in FIG. 6 and FIG. 8, comprises the first plurality of tines (310′ and 310″) the second plurality of tines (410′ and 410″) and the intersection channel 711′ of the flow channel 700′. Upper portions of the pluralities of tines (310″ and 410″) are formed in the upper substrate 30, and therefore may have or provide a height or a portion of the tine that is greater in height than a height of the intersection channel 711′, and the height or portion of the tine extends across the intersection channel 711′. The sheath fluid channel structures (230′ and 240′) and the associated pluralities of tines (310′ and 410′) correspond to the portions (230″ and 240″, and 310″ and 410″ respectively) in the upper substrate 30 to form or provide the full upper, lower, and side surfaces for the tines. A bottom surface of the upper substrate 30, when joined to the lower substrate 20, forms the top of the sample channel 500′, intersection channel 711′, flow channel 700′, interrogation region 800′, and outlet region 900′.

Relatively upstream of the intersection (600′ and 600″), the sample fluid channel 500′ comprises geometric features or restrictions 522′ and 520′, which may be ramps and/or tapers, and further comprises a sample fluid outlet or introduction point 524′ which provides for the flow of sample fluid from the sample fluid channel 500′ into the intersection channel 711′. The intersection channel 711′ is a portion of the flow channel 700′. Relatively downstream of the intersection (600′ and 600″) the flow channel 700′ may lead to one or more geometric features or restrictions, such as the restriction 720′. An interrogation region 800′ provides for interrogation or inspection of sample fluid or particles disposed therein and for action on said particles. An expansion region 910′ in the outlet region 900′ widens to provide for sheath or buffer fluid and sample fluid streams to flow out through outlet channels 920′, 930′, and 940′ and corresponding outlets 922′, 932′, and 942′.

With reference now to FIGS. 9-15, various views of a flow simulation of a sample fluid 50 and a sheath or buffer fluid 60 in a microfluidic channel structure 100 are provided. The views provided in FIGS. 9-15 represent the real-world flow of a sample fluid 50 in a laminar flow regime with a sheath or buffer fluid 60 in the microfluidic channel structure 100 through a software simulation, such as in a COMSOL® MULTIPHYSICS® software simulation program.

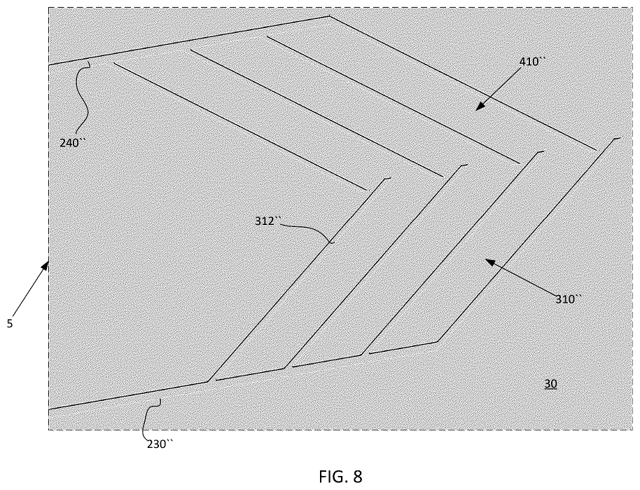

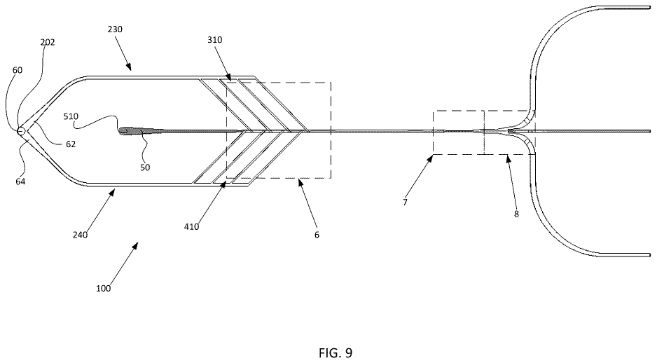

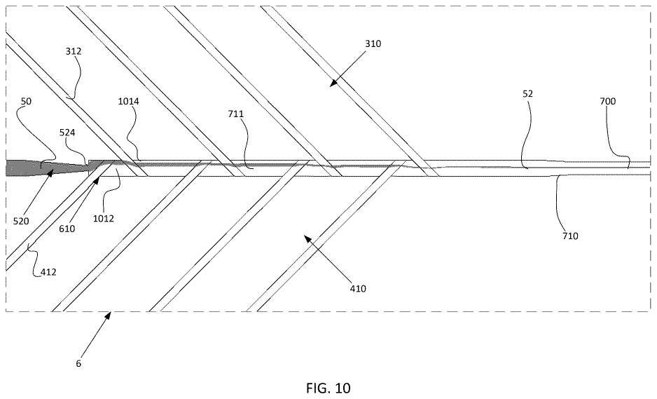

The sample fluid 50 is flowed into a sample fluid inlet 510 and the sample fluid 50 stream flows relatively downstream toward the intersection, shown in detail in region 6 of FIG. 10. and in region 9 of FIG. 14. The sheath fluid is branched off or diverted into two separate flows, a first flow 62 and a second flow 64, which then flow down the respective first 230 and second 240 sheath fluid channels into the corresponding first plurality of tines 300 and second plurality of tines 400 before flowing into the intersection via the tines, such as tines 312 and 412.

In region 6 as shown in FIG. 10 and in region 9 as shown in FIG. 14, the sample fluid 50 flows from the area 520 through the sample fluid outlet or introduction point 524 into the intersection channel 711 of the intersection 600. In the first intersection 610, sheath or buffer fluid 64 from the tine 412 flows into the intersection channel 711 of the intersection 600, diverting or compressing the sample fluid 50 towards the direction of the tine 312 and away from the tine 412. The sheath or buffer fluid 1012 is primarily surrounding the sample fluid 50 from three sides, but may also partially envelop the sample fluid from a fourth side. The sample fluid 50 can be seen directed or compressed primarily in a first lateral direction, but also partially in at least one vertical direction. Subsequent to the introduction of the sheath or buffer fluid 64 from tine 412, the sheath or buffer fluid 62 is introduced via the tine 312. At the introduction of the sheath or buffer fluid 64, the sheath or buffer fluid 1014 in the intersection channel 711 can be seen moving the sample fluid 50 away from the tine 312 and towards the direction of the tine 412. In this manner, the sample fluid 50 is directed or compressed from substantially three sides, but may also be partially enveloped from a fourth side. It can be seen from the detail in region 6 as shown in FIG. 10 and in region 9 as shown in FIG. 14 that the sample fluid 50 is focused asymmetrically. The shape of the fluid flow of the sample fluid 50 resembles that of an “s”, a snake, or a meandering river. The repeated introduction of sheath fluid (62 and 64) from the further downstream tines (e.g., tines 1402, 1404, 1412, 1414, 1422, and 1424) further compresses, narrows, or focuses the sample fluid 50 as it progresses downstream. It is the introduction, including the repeated introduction, of the sheath fluid 60 that provides for a gradual compression, narrowing, or focusing of the sample fluid 50 stream in a manner that resembles, and may confer similar orientation and alignment benefits to, an inertial focusing regime microfluidic chip having a channel design corresponding to the shape of the sample fluid 50 stream.

After asymmetric introduction of the sheath fluid (62 and 64) downstream through the intersection 600, the sample fluid 50 stream is a narrower, compressed, or focused stream 52. The focused stream 52 comprises a narrower, focused stream wherein some degree of order has been conferred on the randomly oriented particles in the stream. Additional orientation, conditioning, alignment, and positioning of both the particles within the focused stream 52 and the focused stream 52 itself occur as the focused stream 52 flows downstream through the length of the flow channel 700 and through one or more restrictions or geometric features, such as the restriction 710 and 720 which comprise ramps or tapers. Additionally, the focused stream 52, after exiting the intersection 600, has been positioned relatively toward the longitudinal centerline of the flow channel 700, and is positioned relatively above the centerline 700 in a vertical direction. Specifically, when the focused stream 52 reaches the interrogation region 800, shown in detail in region 7 in FIG. 11 and in region 10 of FIG. 15, the focused stream 52 will be optimally shaped and conditioned such that any particles suspended or disposed within the focused stream 52 may be interrogated or identified. For example, the stream 52 may be positioned to be in the optimal location to be within the focal plane of a laser and may have a width and particle velocity variation that are small enough to permit individual interrogation of particles within the focused stream 52. In one embodiment, the stream position is offset 10-30 microns from either the centerline or top surface, the stream position horizontally is within 50 microns of the centerline, the stream width is 2-100 microns, and the particle velocity is approximately 5-15 m/s at a sheath or buffer fluid flow rate of 2-4 mL/min.

As shown in region 8 of FIG. 12, after interrogation the focused stream 52 enters an expansion region 910 of the outlet region 900 and flows out through the outlet channel 920. The sheath or buffer fluid 60 may flow out through one or more of the channels 920, 930, and 940 depending on the flow conditions.

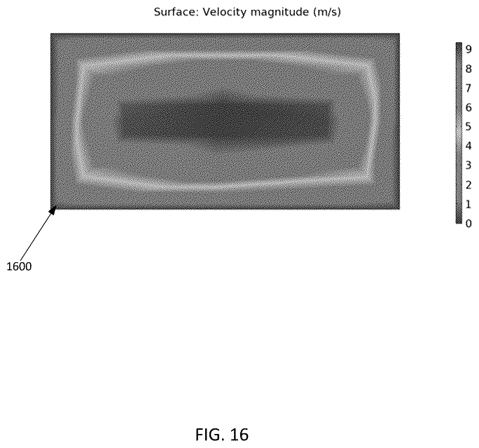

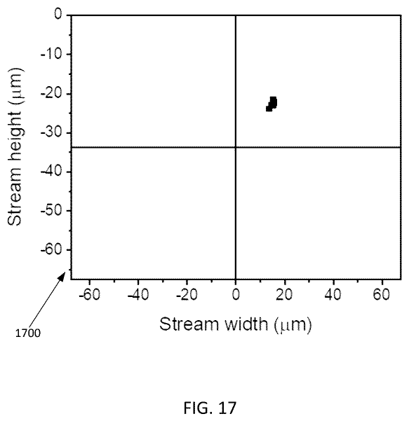

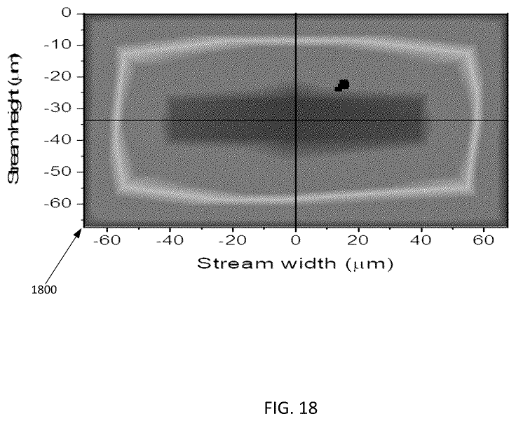

With reference now to FIGS. 16-18, the velocity profile for fluid flows within the microfluidic channel structure 100 and the simulated position of particles within the fluid flow at the interrogation region 800 are provided. The graphs shown in FIGS. 16-18 represent the flow conditions in a channel size of 135 microns and 67.5 microns in height. In the velocity magnitude graph 1660, it can be seen that the magnitude of velocity of the fluid flow within the channel increases inwardly towards the center of the channel with the velocity being near 0 m/s at the edges and greater than 9 m/s towards the center of the channel. It can also be appreciated that there is a gradient of velocity that increases towards the center of the channel. In the stream size graph 1700, the relative size and position of a sample stream, such as the focused stream 52 shown in FIGS. 9-15, is provided. The location of the stream is offset by approximately 20 microns from a top surface, and approximately 15 microns from the centerline horizontally. The size of the stream is approximately 5-10 microns in width and height. The graph 1800 overlays the stream position shown in the graph 1700 with the velocity gradient shown in graph 1600. It can be seen that the position of the sample fluid stream is at the border of a velocity gradient, and is not positioned entirely within a portion of the stream having a uniform velocity.

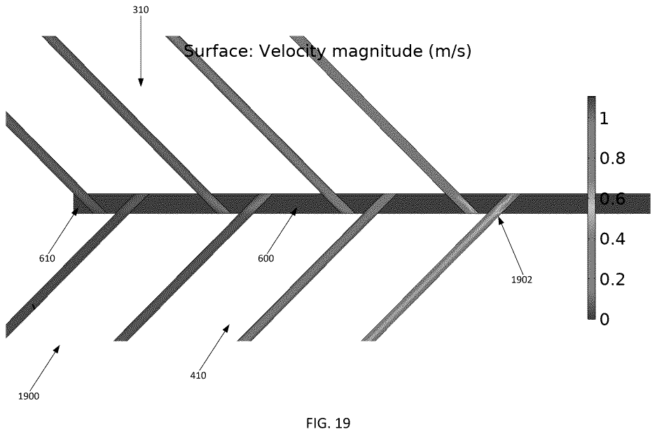

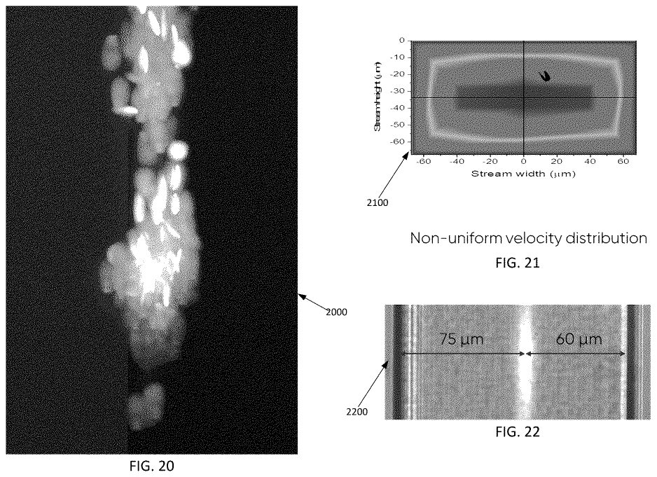

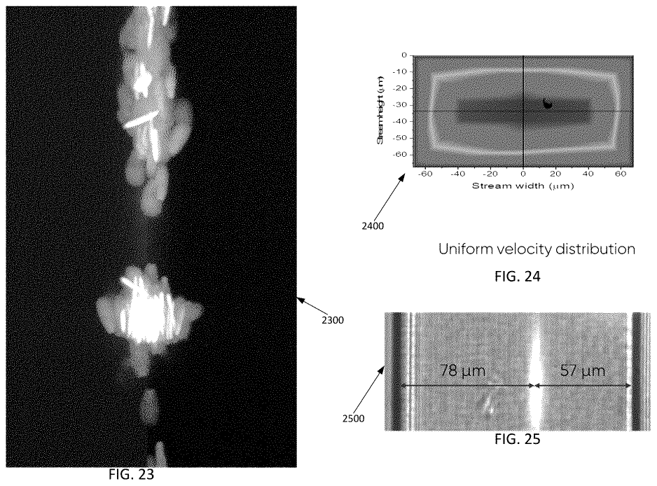

A non-uniform velocity profile for the sample fluid stream would lend to particles within the stream having varying, or non-uniform, velocities. For example, if attempting to interrogate particles at a first location and acting on particles at a second location downstream of the first location, the velocity of the particles may vary by +/−2 m/s and the position of the particles at the second location may vary by 1-15 microns, depending on how far downstream the second location is from the first location. Minimizing the velocity variation is important to accurate measurement and action on the particles. Therefore, it is desirable to obtain a more uniform velocity profile for the sample stream and particles therein. This can be achieved by varying the velocity, rate, or volume of flow of sheath fluid into the intersection from the pluralities of tines. Varying these parameters can be achieved by changing the ratio of the portion of the tine that is above and below the flow channel at the intersection, and by changing the relative length or cross-sectional area of the tines. A comparison of a flow stream having a non-uniform velocity (FIGS. 20-22) and a uniform velocity (FIGS. 23-25) is shown. Additionally, the graph 1900 in FIG. 19 illustrates the difference in the surface velocity magnitude in the tines of the microfluidic channel structure between the first intersection or junction 610 where the velocity is relatively higher and at the end of the intersection area 1902 where the velocity is relatively lower.

With reference now to the graphs in FIGS. 20-22 and in FIGS. 23-25, a comparison of a flow stream having a non-uniform velocity (FIGS. 20-22) and a uniform velocity (FIGS. 23-25) is provided.

FIG. 20 provides a captured stroboscopic image 2000 of approximately two thousand (2000) particles (bovine sperm cells) captured at a fixed point in the interrogation region of the asymmetric microfluidic channel structure. The sample stream, as shown in the velocity gradient graph 2100 of FIG. 21 and in the microscopic image 2200 in FIG. 22, is positioned approximately 15 microns offset from the longitudinal centerline and approximately 22 microns from the top surface of the channel. In this position, the fluid stream is not at a uniform velocity, and therefore the sample stream is subjected to a velocity gradient.

FIG. 23 provides a captured stroboscopic image 2300 of approximately two thousand (2000) particles (bovine sperm cells) captured at a fixed point in the interrogation region of the asymmetric microfluidic channel structure. The sample stream, as shown in the velocity gradient graph 2400 of FIG. 24 and in the microscopic image 2500 in FIG. 25, is positioned approximately 15 microns offset from the longitudinal centerline and approximately 30 microns from the top surface of the channel. In this position, the fluid stream is at a uniform velocity, and therefore the sample stream is subjected to a relatively constant velocity (low velocity gradient) at that location within the channel.

The stroboscopic image 2000 illustrates that the particles are not all arriving at a fixed point at the same time, and it can be seen that the position of the particles is distributed or spread vertically down the image instead of being substantially more concentrated at a single point, as is provided in the stroboscopic image 2300. This is a result of the particles travelling at varying velocities and arriving at the fixed point at different times in the image 2000—different distances from the fixed point, compared to the relatively more constant velocity shown in the image 2300. The offset shown in FIGS. 20-22 is a result of the value of the height of the tines above the height of the intersection channel being lower than for the chip illustrated in FIGS. 23-25, or it could be said that ratio of the portion of the tine above the intersection channel to the portion of the tine below the intersection channel is lower for the chip illustrated in FIGS. 20-22 than for the chip illustrated in FIGS. 23-25. As shown in FIGS. 20-22 the top portion is 50 microns to an overall tine height of 400 microns, and in FIGS. 23-25 the top portion is 100 microns to an overall tine height of 400 microns.

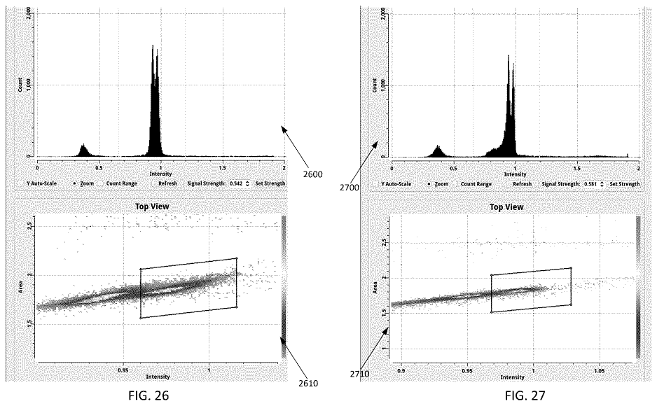

With reference now to FIGS. 26 and 27, count/intensity (2600, 2700) and area/intensity (2610, 2710) histograms are provided for the fluorescence intensity of the emitted fluorescence of bovine sperm cells flowing through the interrogation region of an asymmetrically focusing microfluidic channel structure. In FIG. 27 it can be seen that the ratio of peak to valley (resolution) in the graph 2700 is greater that the ratio of peak to valley in the graph 2600. The position of the peak/area output in the graph 2710 is also relatively less offset than the output shown in the graph 2610. This is indicative of the position of the sample stream in FIG. 27 compared to FIG. 26. In FIG. 26, the sample stream is more offset and in a position within the channel such that it is subject to a greater velocity gradient and has a larger velocity deviation due to the non-uniform velocity of the stream and its associated particles. In FIG. 27, the sample stream is less offset and in a position within the channel such that it is subject to a relatively lower velocity gradient and has a low velocity deviation due to the substantially uniform velocity of the stream and its associated particles. It can therefore be seen that it is preferable to achieve a uniform or substantially uniform velocity for interrogation and identification of particles.

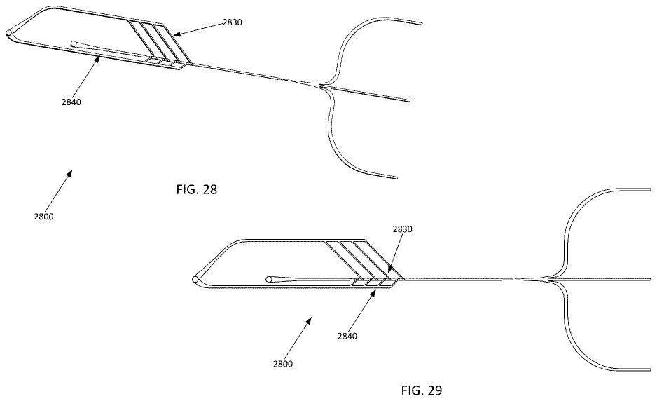

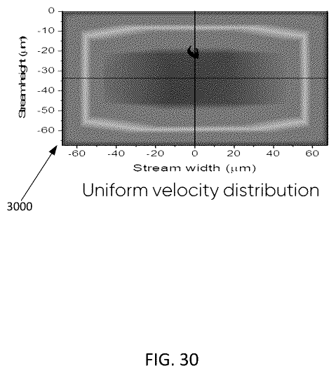

With reference now to FIGS. 28-29, 31-32, and 35, what are provided are alternate embodiments of asymmetric focusing microfluidic channel structures, such as those provided in the dimensions shown in TABLE 1, and associated velocity gradient graphs in FIGS. 30, 33-34, and 36. The asymmetric focusing microfluidic channel structures shown in FIGS. 28-29, 31-32, and 35, achieve the same function as the structure 100 shown in FIG. 1, however, variations in tine length, tine cross-sectional area, intersection channel cross-sectional area, flow channel length, flow channel cross-sectional area, number and type of restrictions or geometric features in the flow channel (e.g., the restrictions 710 and 720 shown in FIG. 1), and cross-sectional area of the interrogation region are used to determine the position and flow characteristics of the sample fluid stream and the velocity, position, and orientation of particles therein. Additionally, varying the portion of each tine that is disposed above the intersection channel is used to set the vertical position or offset of the fluid stream within the flow channel, especially at the interrogation region.

For example, in one embodiment as shown in the microfluidic channel structure 2800 of FIGS. 28 and 29, the length of each tine in the set of tines 2840 is less than the length of each tine in the set of tines 2830. This provides for relatively higher flow velocity or flow rate of sheath or buffer fluid from the set of tines 2830 than from the tines 2840, thereby positioning the sample fluid stream relatively in the center of the channel as shown in the graph 3000 in FIG. 30. This achieves a uniform velocity distribution for the sample fluid stream and particles positioned therein.

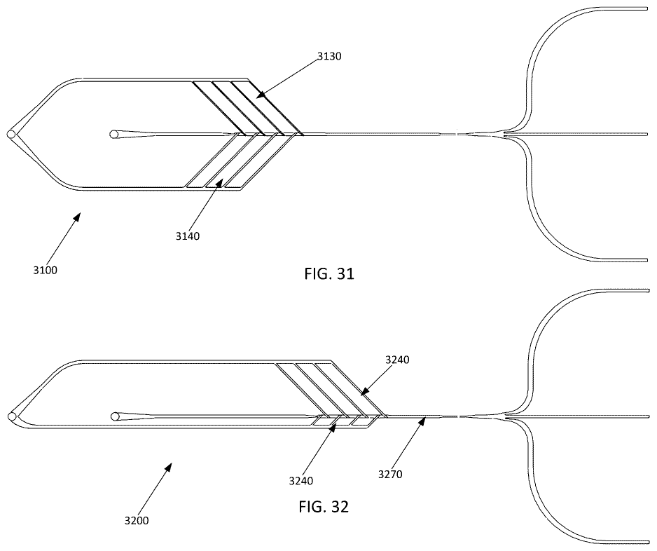

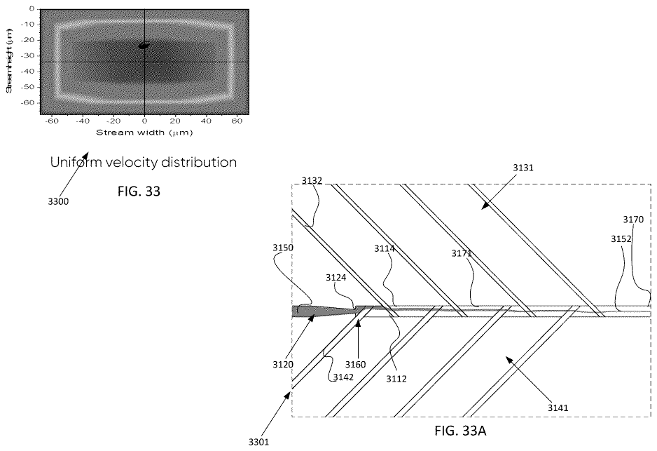

In another embodiment, as shown in the microfluidic channel structure 3100 of FIG. 31, the width (and cross-sectional area) of each tine in the set of tines 3130 is less than the width of each tine in the set of tines 3140. This provides for relatively higher flow velocity or flow rate of sheath or buffer fluid from the set of tines 3130 than from the tines 3140, thereby positioning the sample fluid stream relatively in the center of the channel as shown in the graph 3300 in FIG. 33. This achieves a uniform velocity distribution for the sample fluid stream and particles positioned therein.

With reference now to FIG. 33A, and also to FIGS. 31 and 33, a plan view of a flow simulation of a sample fluid 3150 and a sheath or buffer fluid in a microfluidic channel structure 3100 intersection 3301 is provided. The view provided in FIG. 33A represents the real-world flow of a sample fluid 3150 in a laminar flow regime with a sheath or buffer fluid (e.g., 3112, 3114) in the microfluidic channel structure 3100 through a software simulation, such as in a COMSOL® MULTIPHYSICS® software simulation program.

The sample fluid 3150 is flowed into a sample fluid inlet and the sample fluid 3150 stream flows relatively downstream toward the intersection 3301. The sheath fluid is branched off or diverted into two separate flows, a first flow and a second flow, which then flow down the respective first and second sheath fluid channels into the corresponding first plurality of tines 3130 and second plurality of tines 3140 before flowing into the intersection via the tines, such as tines 3132 and 3142.

In the intersection 3301 the sample fluid 3150 flows from the area 3120 through the sample fluid outlet or introduction point 3124 into the intersection channel 3171 of the intersection 3301. In the first intersection 3160, sheath or buffer fluid from the tine 3142 flows into the intersection channel 3171 of the intersection 3301, diverting or compressing the sample fluid 3150 towards the direction of the tine 3132 and away from the tine 3142. The sheath or buffer fluid 3112 is primarily surrounding the sample fluid 3150 from three sides, but may also partially envelop the sample fluid from a fourth side. The sample fluid 3150 can be seen directed or compressed primarily in a first lateral direction, but also partially in at least one vertical direction. Subsequent to the introduction of the sheath or buffer fluid from tine 3142, the sheath or buffer fluid is introduced via the tine 3132. At the introduction of the sheath or buffer fluid, the sheath or buffer fluid 3114 in the intersection channel 3171 can be seen moving the sample fluid 3150 away from the tine 3132 and towards the direction of the tine 3142. In this manner, the sample fluid 3150 is directed or compressed from substantially three sides, but may also be partially enveloped from a fourth side. It can be seen that the sample fluid 3150 is focused asymmetrically.

The shape of the fluid flow of the sample fluid 3150 resembles that of an “s”, a snake, or a meandering river. The repeated introduction of sheath fluid from the further downstream tines further compresses, narrows, or focuses the sample fluid 3150 as it progresses downstream. It is the introduction, including the repeated introduction, of the sheath fluid that provides for a gradual compression, narrowing, or focusing of the sample fluid 3150 stream in a manner that resembles, and may confer similar orientation and alignment benefits to, an inertial focusing regime microfluidic chip having a channel design corresponding to the shape of the sample fluid 3150 stream.

After asymmetric introduction of the sheath fluid downstream through the intersection 3301, the sample fluid 3150 stream is a narrower, compressed, or focused stream 3152. The focused stream 3152 comprises a narrower, focused stream wherein some degree of order has been conferred on the randomly oriented particles in the stream. Additional orientation, conditioning, alignment, and positioning of both the particles within the focused stream 3152 and the focused stream 3152 itself occurs as the focused stream 3152 flows downstream through the length of the flow channel 3170 and through one or more restrictions or geometric features, which may comprise ramps or tapers. Additionally, the focused stream 3152, after exiting the intersection 3301, has been positioned relatively toward the longitudinal centerline of the flow channel 3170, and is positioned relatively above the centerline 3170 in a vertical direction. Specifically, the focused stream 3152 is relatively more centered and within a location of the channel, as shown in the velocity gradient cross-section 3300 of FIG. 33, where the velocity gradient is small, or wherein the velocity is relatively uniform.

Compared to the simulation shown in FIGS. 9-14 for the asymmetric microfluidic channel structure 100, the simulation shown in FIG. 33A of the asymmetric microfluidic channel structure 3100 illustrates the positioning that may be achieved through the use of unequal tine width, and through the use of different amounts of channel height above the intersection channel (respectively 711 and 3171). The tines 3131 of the asymmetric microfluidic channel structure 3100 are relatively narrower than the tines 3141 of the same structure (compared to, for example, the tines 310 and 410 of the asymmetric microfluidic channel structure 100 which are of equal width). Specifically, the focused stream 3152 is relatively more in the center of the channel than the focused stream 52, and a more significant, or greater, portion of the focused stream 3152 is in a uniform (or low velocity gradient) flow location within the channel in a vertical direction.

In one embodiment, the stream position is offset 10-30 microns from either the centerline or top surface, the stream position horizontally is within 20 microns of the centerline, the stream width is 2-100 microns, and the particle velocity is approximately 5-15 m/s at a sheath or buffer fluid flow rate of 2-4 mL/min.

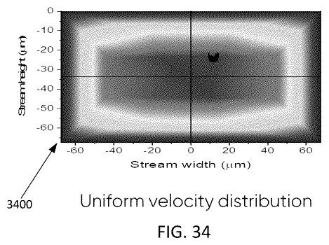

In another embodiment, as shown in the microfluidic channel structure 3200 of FIG. 32, the length of each tine in the set of tines 3240 is less than the length of each tine in the set of tines 3230. This provides for relatively higher flow velocity or flow rate of sheath or buffer fluid from the set of tines 3230 than from the tines 3240, thereby positioning the sample fluid stream somewhat towards the center of the channel. However, the height of the flow channel 3270 is also relatively smaller than, for example, the flow channel 700 shown in FIG. 1, and therefore the centering effect is reduced and the sample stream remains somewhat offset as shown in the graph 3400 in FIG. 34. This achieves a non-uniform velocity distribution for the sample fluid stream and particles positioned therein that may be detrimental to interrogation, identification, and action on the particles.

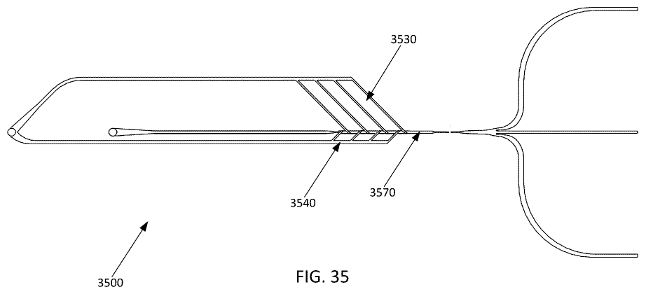

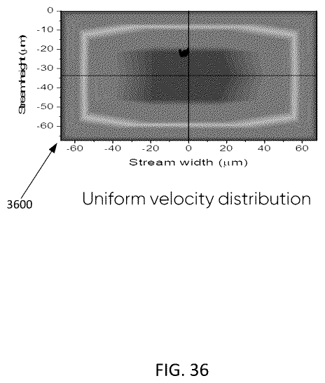

In another embodiment, as shown in the microfluidic channel structure 3500 of FIG. 35, the length of each tine in the set of tines 3540 is less than the length of each tine in the set of tines 3530. This provides for relatively higher flow velocity or flow rate of sheath or buffer fluid from the set of tines 3530 than from the tines 3540, thereby positioning the sample fluid stream somewhat towards the center of the channel. However, the height and length of the flow channel 3570 is also relatively smaller and shorter than, for example, the flow channel 700 shown in FIG. 1, and therefore unlike the microfluidic channel structure 3200 the centering effect is not reduced and the sample stream is centered as shown in the graph 3600 in FIG. 36. This achieves a uniform velocity distribution for the sample fluid stream and particles positioned therein that is beneficial to interrogation, identification, and action on the particles.

In one embodiment, what is provided is a microfluidic channel structure comprising: a sheath fluid channel structure originating upstream at a sheath fluid inlet, the sheath fluid channel structure comprising a first side channel structure and a second side channel structure; a sample fluid channel originating at an upstream sample fluid inlet; a flow channel disposed downstream of the sample fluid inlet and the sheath fluid inlet; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In various embodiments, the intersection of the plurality of tines with the flow channel is after a sample introduction location.

In various embodiments, the intersection of the plurality of tines with the flow channel is staggered.

In various embodiments, the intersection of the plurality of tines with the flow channel alternates between an intersection of a tine from the first side channel structure and an intersection of a tine from the second side channel structure.

In various embodiments, the plurality of tines comprises a set of four tines.

In various embodiments, the plurality of tines intersects the flow channel at an angle between 1 and 90 degrees.

In various embodiments, the plurality of tines intersects the flow channel at an angle of 45 degrees.

In various embodiments, a height of the sheath fluid channel structure is larger than a height of the flow channel.

In various embodiments, a height of the tines is larger than a height of the flow channel.

In various embodiments, the intersection of each tine in the plurality of tines extends across the flow channel from a first side of the flow channel to a second side of the flow channel.

In various embodiments, a first portion of the sheath fluid channel structure is disposed in a first substrate layer and a second portion of the sheath fluid channel structure is disposed in a second substrate layer.

In various embodiments, a portion of each tine in the plurality of tines for each of the first side channel structure and second side channel structure is disposed above and through a top surface of the flow channel.

In various embodiments, a portion of each tine in the plurality of tines for each of the first side channel structure and second side channel structure is disposed below and through a bottom surface of the flow channel.

In various embodiments, a length of each tine in the plurality of tines of the second side channel structure is shorter than that of each tine in the plurality of tines of the first side channel structure.

In various embodiments, a length of each tine in the plurality of tines of the first side channel structure is longer than that of each tine in the plurality of tines of the second side channel structure.

In various embodiments, a length of each tine in the plurality of tines of the second side channel structure is longer than that of each tine in the plurality of tines of the first side channel structure.

In various embodiments, a length of each tine in the plurality of tines of the first side channel structure is shorter than that of each tine in the plurality of tines of the second side channel structure.

In various embodiments, a width of each tine in the plurality of tines of the second side channel structure is wider than that of each tine in the plurality of tines of the first side channel structure.

In various embodiments, a width of each tine in the plurality of tines of the first side channel structure is narrower than that of each tine in the plurality of tines of the second side channel structure.

In various embodiments, a width of each tine in the plurality of tines of the second side channel structure is narrower than that of each tine in the plurality of tines of the first side channel structure.

In various embodiments, a width of each tine in the plurality of tines of the first side channel structure is wider than that of each tine in the plurality of tines of the second side channel structure.

In various embodiments, the sample fluid channel intersects the flow channel upstream of the intersection of the sheath fluid channel structure and the flow channel.

In various embodiments, a height of the sample fluid channel is equal to a height of the flow channel.

In various embodiments, the flow channel comprises a first geometric focusing region.

In various embodiments, the flow channel comprises a second geometric focusing region.

In various embodiments, the first geometric focusing region comprises a taper.

In various embodiments, the first geometric focusing region comprises a ramp.

In various embodiments, the first geometric focusing region comprises a ramp and a taper.

In various embodiments, the second geometric focusing region comprises a taper.

In various embodiments, the second geometric focusing region comprises a ramp.

In various embodiments, the second geometric focusing region comprises a ramp and a taper.

In various embodiments, the flow channel comprises a detection region.

In various embodiments, a height of the detection region is less than a height of an upstream portion of the flow channel.

In various embodiments, a width of the detection region is less than a width of an upstream portion of the flow channel.

In various embodiments, the detection region is disposed downstream of an intersection of the sheath fluid channel structure and the flow channel.

In various embodiments, the microfluidic channel structure further comprises an expansion region.

In various embodiments, the expansion region is disposed downstream of the detection region.

In various embodiments, a height of the expansion region is larger than a height of an upstream portion of the flow channel.

In various embodiments, a height of the expansion region is larger than a height of the detection region.

In various embodiments, the microfluidic channel structure, further comprises a set of outlet channels.

In various embodiments, the set of outlet channels comprises a single outlet.

In various embodiments, the set of outlet channels comprises three outlets.

In various embodiments, a ratio of widths of the set of outlet channels is 1:2:1.

In various embodiments, the set of outlet channels is disposed downstream of the detection region.

In various embodiments, the set of outlet channels comprise a primary outlet channel and a set of secondary outlet channels.

In various embodiments, the set of secondary outlet channels comprise a set of waste channels.

In various embodiments, the intersection is configured to asymmetrically focus particles in a sample stream flowing through the flow channel.

In various embodiments, the intersection is configured to asymmetrically focus and orient particles in a sample stream flowing through the flow channel.

In various embodiments, the particles are cells.

In various embodiments, the cells are sperm cells.

In various embodiments, the intersection is configured to asymmetrically focus a sample stream.

In various embodiments, the asymmetric focusing comprises flowing a first sheath fluid from a first tine in the plurality of tines of the first side channel structure into the flow channel to direct a sample stream in the flow channel towards a first side wall opposite of the first tine, and flowing a second sheath fluid from a second tine in the plurality of tines of the second side channel structure into the flow channel to direct the sample stream towards a second side wall opposite of the second tine.

In various embodiments, the plurality of tines of the first side channel structure is offset relative to the plurality of tines of the second side channel structure to provide for the asymmetric focusing of the sample stream.

In various embodiments, the asymmetric focusing comprises surrounding the sample stream with sheath fluid from substantially three directions to cause the sample stream to move towards a fourth direction, and subsequently surrounding the sample stream with sheath fluid from substantially three directions to cause the sample stream to move away from the fourth direction.

In various embodiments, the asymmetric focusing comprises repeatedly directing the sample stream towards and away from the fourth direction by repeatedly surrounding the sample stream with sheath fluid from at least three directions.

In various embodiments, the sheath fluid channel structure comprises a herringbone structure configuration.

In various embodiments, each tine in the plurality of tines for the first and second side channel structures comprises a bone of a herringbone structure configuration.

In various embodiments, a width of the main sheath fluid channel is from 50-1000 microns.

In various embodiments, a width of the main sheath fluid channel is 300 microns.

In various embodiments, a height of the main sheath fluid channel is from 100-500 microns.

In various embodiments, a height of the main sheath fluid channel is 400 microns.

In various embodiments, a length of the main sheath fluid channel is from 5000-35000 microns.

In various embodiments, a length of the main sheath fluid channel is 15311 microns.

In various embodiments, a length of the main sheath fluid channel is 24770 microns.

In various embodiments, a width of a tine in the plurality of tines for the first and second side channel structures is from 50-500 microns.

In various embodiments, a width of a tine in the plurality of tines for the first and second side channel structures is 150 microns.

In various embodiments, a width of a tine in the plurality of tines for the first side channel structure is 150 microns and a width of a tine in the plurality of tines for the second side channel structure is 105 microns.

In various embodiments, a width of a tine in the plurality of tines for the first side channel structure and a width of a tine in the plurality of tines for the second side channel structure are not equal.

In various embodiments, a height of a tine in the plurality of tines for the first and second side channel structures is from 100-500 microns.

In various embodiments, a height of a tine in the plurality of tines for the first and second side channel structures is 400 microns.

In various embodiments, a height of a tine in the plurality of tines for the first and second side channel structures is 465 microns.

In various embodiments, a height of a tine in the plurality of tines for the first and second side channel structures is 475 microns.

In various embodiments, a height of a tine in the plurality of tines for the first and second side channel structures is 490 microns.

In various embodiments, a height of a tine in the plurality of tines for the first and second side channel structures comprises 50 microns in a first substrate layer and 350 microns in a second substrate layer.

In various embodiments, a height of a tine in the plurality of tines for the first and second side channel structures comprises 65 microns in a first substrate layer and 400 microns in a second substrate layer.

In various embodiments, a height of a tine in the plurality of tines for the first and second side channel structures comprises 75 microns in a first substrate layer and 400 microns in a second substrate layer.

In various embodiments, a height of a tine in the plurality of tines for the first and second side channel structures comprises 100 microns in a first substrate layer and 300 microns in a second substrate layer.

In various embodiments, a height of a tine in the plurality of tines for the first and second side channel structures comprises 90 microns in a first substrate layer and 400 microns in a second substrate layer.

In various embodiments, a length of a tine in the plurality of tines for the first and second side channel structures is from 1000-15000 microns.

In various embodiments, a length of tines in the plurality of tines for the first side channel structure and a length of tines in the plurality of tines for the second side channel structure are not equal.

In various embodiments, a length of tines in the plurality of tines for the first side channel structure is 1407 microns and a length of tines in the plurality of tines for the second side channel structure is 7460 microns.

In various embodiments, a length of a tine in the plurality of tines for the first and second side channel structures is 7460 microns.

In various embodiments, a distance between a tine in the plurality of tines for the first side channel structure and a tine in the plurality of tines for the second side channel structure at a first side wall of the flow channel is from 600-1500 microns.

In various embodiments, a distance between a tine in the plurality of tines for the first side channel structure and a tine in the plurality of tines for the second side channel structure at a first side wall of the flow channel is 820 microns.

In various embodiments, a width of the flow channel is from 50-500 microns.

In various embodiments, a width of the flow channel is 300 microns.

In various embodiments, a width of the flow channel is 250 microns.

In various embodiments, a width of the flow channel is 300 microns in a first region downstream of the intersection and 250 microns at a second region downstream of the intersection.

In various embodiments, a height of the flow channel is from 100-500 microns.

In various embodiments, a height of the flow channel is 150 microns.

In various embodiments, a height of the flow channel is 67.5 microns.

In various embodiments, a length of the flow channel is from 1001-30000 microns.

In various embodiments, a length of the flow channel is 9200 microns.

In various embodiments, a length of the flow channel is 10912 microns.

In various embodiments, a length of the flow channel is 20112 microns.

In various embodiments, a length of the flow channel is 4912 microns.

In various embodiments, a length of the flow channel is 2319 microns.

In various embodiments, a length of the flow channel is 7231 microns.

In various embodiments, an interrogation region of the flow channel comprises a width of 50-200 microns.

In various embodiments, an interrogation region of the flow channel comprises a width of 135 microns.

In various embodiments, an interrogation region of the flow channel comprises a height of 50-150 microns.

In various embodiments, an interrogation region of the flow channel comprises a height of 67.5 microns.

In various embodiments, an interrogation region of the flow channel comprises a length from 100-5000 microns.

In various embodiments, an interrogation region of the flow channel comprises a length of 2319 microns.

In various embodiments, a sample fluid stream is positioned at a set height within the flow channel.

In various embodiments, a height of the plurality of tines of both the first and second microfluidic channel structure flows a sheath fluid stream into the flow channel to position the sample fluid stream at the set height within the flow channel.

In various embodiments, the set height is at ⅔ of a height of the flow channel, +/−⅛ of the height of the flow channel.

In various embodiments, the set height is at ⅔ of a height of the flow channel.

In various embodiments, the set height is at ⅔ of a height of the flow channel, +/−⅛ of the height of the flow channel.

In various embodiments, the set height is at ¾ of the height of the flow channel.

In various embodiments, the set height is not at a centerline of the flow channel.

In various embodiments, the set height is offset from a centerline of the flow channel.

In various embodiments, the set height is from 18-25 microns from a top surface of the flow channel.

In various embodiments, the set height is at 18 microns from a top surface of the flow channel.

In various embodiments, the set height is at 19 microns from a top surface of the flow channel.

In various embodiments, the set height is at 20 microns from a top surface of the flow channel.

In various embodiments, the set height is at 21 microns from a top surface of the flow channel.

In various embodiments, the set height is at 22 microns from a top surface of the flow channel.

In various embodiments, the set height is at 23 microns from a top surface of the flow channel.

In various embodiments, the set height is at 24 microns from a top surface of the flow channel.

In various embodiments, the set height is at 25 microns from a top surface of the flow channel.

In various embodiments, the set height is at least 18 microns from a top surface of the flow channel and is offset from a centerline of the flow channel.

In another embodiment, what is provided is microfluidic chip comprising: a first substrate layer and a second substrate layer; a sheath fluid channel structure originating upstream at a sheath fluid inlet, the sheath fluid channel structure comprising a first side channel structure and a second side channel structure, wherein an upper portion of the first side channel structure and the second side channel structure are disposed in the first substrate layer, and wherein a lower portion of the first side channel structure and the section side channel structure are disposed in the second substrate layer; a sample fluid channel originating at an upstream sample fluid inlet; a flow channel disposed downstream of the sample fluid inlet and the sheath fluid inlet; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In one embodiment, what is provided is a microfluidic chip comprising the microfluidic channel structure of any of the above embodiments.

In one embodiment, what is provided is a flow cytometry system comprising: an interrogation light source; a detector; and a microfluidic chip, the microfluidic chip comprising: a sheath fluid channel structure originating upstream at a sheath fluid inlet, the sheath fluid channel structure comprising a first side channel structure and a second side channel structure; a sample fluid channel originating at an upstream sample fluid inlet; a flow channel disposed downstream of the sample fluid inlet and the sheath fluid inlet; an interrogation region disposed downstream of the flow channel; wherein the first side channel structure and the second side channel structure each comprise a plurality of tines extending from a main sheath fluid channel to the flow channel; wherein the plurality of tines of both of the first channel structure and second channel structure intersect the flow channel at an intersection.

In one embodiment, what is provided is a flow cytometry system comprising the microfluidic chip of any of the above embodiments.

In one embodiment, what is provided is a flow cytometry system comprising a microfluidic chip which comprises the microfluidic channel structure of any of any of the above embodiments.

In one embodiment, what is a method for focusing a sample fluid stream, the method comprising: introducing a sheath fluid as a sheath fluid stream into a sheath fluid channel structure at a sheath fluid inlet; diverting the sheath fluid stream into both a first side channel structure and a second side channel structure of the sheath fluid channel structure; introducing a sample fluid as a sample fluid stream into a sample fluid channel at a sample fluid inlet; flowing the sheath fluid stream into a plurality of tines of the first side channel structure; flowing the sheath fluid stream into a plurality of tines of the second side channel structure; flowing the sample fluid stream into a flow channel disposed downstream of the sample fluid inlet and the sheath fluid inlet; intersecting the sample fluid stream in the flow channel at an intersection with the sheath fluid stream from the plurality of tines of the first side channel structure and from the plurality of tines of the second side channel structure; and asymmetrically focusing the sample fluid stream by the sheath fluid stream at the intersection, wherein the sample fluid stream is focused into a smaller, narrower stream by asymmetric introduction of the sheath fluid stream from the plurality of tines of the first side channel structure and from the plurality of tines of the second side channel structure.

In one embodiment, what is a method for focusing a sample fluid stream, the method comprising: asymmetrically focusing the sample fluid stream by alternately intersecting the sample fluid stream in a flow channel by repeated introduction of a sheath fluid stream.

In one embodiment, what is a method for focusing a sample fluid stream comprising asymmetrically focusing the sample fluid stream in the microfluidic channel structure of any of the above embodiments.

In one embodiment, what is a method for focusing a sample fluid stream comprising asymmetrically focusing the sample fluid stream in a microfluidic chip comprising the microfluidic channel structure of any of any of the above embodiments.

In one embodiment, what is provided is a method for focusing a sample stream, the method comprising: positioning the sample stream at a position that is offset from a centerline of a flow channel.

In various embodiments, the method further comprises positioning the sample stream using the microfluidic channel structure of any of the above embodiments.

In one embodiment, a flow cytometry system is provided which comprises an interrogation apparatus. Although a laser may be used, it is understood that other suitable radiation sources may be used, such as a light emitting diode (LED), arc lamp, etc. to emit a beam which excites the particles. In another embodiment, the light beam can be delivered to the particles by an optical fiber that is embedded in the microfluidic chip at the opening.

In some embodiments, a high intensity laser beam from a suitable laser of a preselected wavelength-such as a 355 nm continuous wave (CW) (or quasi-CW) laser, or a 355 nm pulse laser—is required to excite the particles in the fluid mixture (i.e., sperm cells). The laser emits a laser beam through the window so as to illuminate the particles flowing through the interrogation region of the chip. Since the laser beam can vary in intensity widthwise along the micro-channel, with the highest intensity generally at the center of the flow channel (e.g., midsection of the channel width) and decreasing therefrom, it is imperative that the microfluidic channel structure, including the intersection, focuses the particles at or near the center of the fluid stream where optimal illumination occurs at or near the center of the illumination laser spot. Without wishing to be bound to a particular belief, this can improve accuracy of the interrogation and identification process.

In some embodiments, the high intensity beam interacts with the particles such that the emitted light, which is induced by the beam, is received by an objective lens. The objective lens may be disposed in any suitable position with respect to the microfluidic chip. In one embodiment, the emitted light received by the objective lens is converted into an electronic signal by an optical sensor, such as a photomultiplier tube (PMT) or photodiode, etc. The electronic signal can be digitized by an analog-to-digital converter (ADC) and sent to a digital signal processor (DSP) based controller. The DSP based controller monitors the electronic signal and may then trigger a sorting mechanism.

In other embodiments, the interrogation apparatus may comprise a detector such as a photomultiplier tube (PMT), an avalanche photodiode (APD), or a silicon photomultiplier (SiPM). For example, the optical sensor of the interrogation apparatus may be APD, which is a photodiode with substantial internal signal amplification through an avalanche process.

In some embodiments, a piezoelectric actuator assembly may be used to sort the desired particles in the fluid mixture as the particles leave the interrogation area after interrogation. A trigger signal sent to the piezoelectric actuator is determined by the sensor raw signal to activate a particular piezoelectric actuator assembly when the selected component is detected. In some embodiments, a flexible diaphragm made from a suitable material, such as one of stainless steel, brass, titanium, nickel alloy, polymer, or other suitable material with desired elastic response, is used in conjunction with an actuator to push target particles in the micro-channel into an output channel to isolate the target particles from the fluid mixture. The actuator may be a piezoelectric, magnetic, electrostatic, hydraulic, or pneumatic type actuator.

In alternative embodiments, a piezoelectric actuator assembly or a suitable pumping system may be used to pump the sample fluid into the sample fluid channel toward the intersection. The sample piezoelectric actuator assembly may be disposed at a sample inlet. By pumping the sample fluid mixture into the flow channel, a measure of control can be made over the spacing of the particles therein, such that a more controlled relationship may be made between the particles as they enter the flow channel.

Other embodiments of sorting or separating mechanisms that may be used in accordance with the present invention include, but are not limited to, droplet sorters, mechanical separation, fluid switching, acoustic focusing, holographic trapping/steering, and photonic pressure/steering. In a preferred embodiment, the sorting mechanism for sex-sorting of sperm cells comprises laser kill/ablation of selected X-chromosome-bearing sperm cells.

In laser ablation, the laser is activated when an X-chromosome-bearing sperm cell or Y-chromosome-bearing sperm cell is detected during interrogation, depending on which cell type the system is selecting or enriching for. The laser emits a high intensity beam directed at the sperm cell centered within the fluid stream. The high intensity beam is configured to cause DNA and/or membrane damage to the cell, such as by slicing or ablating the cell, thereby causing infertility or killing the sperm cell. As a result, the final product is comprised predominantly of viable X-chromosome-bearing sperm cells or Y-chromosome-bearing sperm cells depending on which cell type the system is selecting or enriching for. In preferred embodiments, a reduction in the cross-sectional area, either vertically, horizontally, or both, of the flow focusing region geometrically compresses the fluid that carries sperm cells. The geometric compression of the fluid centralizes the sperm cells within the fluid such that the sperm cells are focused at or near a center of the flow channel. This may be used in addition to asymmetric focusing at an intersection. Since the laser beam varies in intensity widthwise along the flow channel, with the highest intensity generally at the center of the flow channel and decreasing therefrom, it is imperative that the microfluidic channel structure focuses the sperm cells at or near the center of the fluid stream where the laser beam has the highest intensity to impart maximum damage to the selected sperm cells.

In one embodiment, the particles that are to be isolated, selected, or enriched for include, for example: isolating viable and motile sperm from non-viable or non-motile sperm; isolating sperm by gender, and other sex sorting variations; isolating stem cells from cells in a population; isolating one or more labeled cells from un-labeled cells distinguishing desirable/undesirable traits; sperm cells with different desirable characteristics; isolating genes in nuclear DNA according to a specified characteristic; isolating cells based on surface markers; isolating cells based on membrane integrity (viability), potential or predicted reproductive status (fertility), ability to survive freezing, etc.; isolating cells from contaminants or debris; isolating healthy cells from damaged cells (i.e., cancerous cells) (as in bone marrow extractions); red blood cells from white blood cells and platelets in a plasma mixture; and isolating any cells from any other cellular components, into corresponding fractions; damaged cells, or contaminants or debris, or any other biological materials that are desired to isolated. The particles may be cells or beads treated or coated with, linker molecules, or embedded with a fluorescent or luminescent label molecule(s). The particles may have a variety of physical or chemical attributes, such as size, shape, materials, texture, etc.

In one embodiment, a heterogeneous population of particles may be measured simultaneously, with each component being examined for different quantities or regimes in similar quantities (e.g., multiplexed measurements), or the particles may be examined and distinguished based on a label (e.g., fluorescent), image (due to size, shape, different absorption, scattering, fluorescence, luminescence characteristics, fluorescence or luminescence emission profiles, fluorescent or luminescent decay lifetime), and/or particle position etc.

In one embodiment, a focusing method may be used in order to position the particles for interrogation in the interrogation region. In some embodiments, the particles are pre-stained with dye (e.g., Hoechst dye), in order to allow fluorescence, and for imaging to be detected. Initially, the particles in the sample fluid mixture flow through sample channel and have random orientation and position. At the intersection, the sample mixture flowing in the flow channel is compressed by the sheath or buffer fluids flowing from the sheath channel structure at least horizontally on at least both sides of the flow, and at least vertically on at least both sides of the flow, but in an asymmetric manner. For example, the sample may first be compressed primarily from three directions towards a fourth direction, and then from three directions away from the fourth direction, in an alternating manner. This may comprise directing the sample stream towards a first side and then away from the first side towards a second side of a flow channel in an alternating manner by asymmetric introduction of sheath or buffer fluid. The flow of the sample stream through the intersection as it is asymmetrically focused would resemble a meandering river, wherein the width and/or height of the sample stream is reduced along a flow direction. As a result, the particles are focused and compressed into a thin stream and the particles (e.g., sperm cells) move approximately toward a center of the channel width.

In another embodiment, what is provided are additional geometric focusing or constricting steps where the sample mixture containing the particles is further compressed, at least horizontally, downstream of the intersection. This comprises physical or geometric compression instead of another intersection of sheath fluids. In preferred embodiments, the particles are flowing in approximately single file formation, and in other embodiments the particles are flowing in approximately single file formation with regulated or regular spacing.

Accordingly, the microfluidic chip and microfluidic channel structure described herein may be used in the focusing method described above. In one embodiment, the present invention provides a method of asymmetrically focusing particles in a fluid flow.

Compression of the fluid mixture, by the asymmetric introduction of sheath fluid and/or the physical structures at the constricting and focusing regions constricts the particles of the fluid mixture into a relatively smaller, narrower stream bounded by the sheath fluids. For example, sheath fluid asymmetrically introduced into the intersection by tines of the sheath fluid channel structure compresses the sample fluid stream from all sides into a relatively smaller, narrower stream while maintaining laminar flow. Flow of the sample fluid and sheath fluids in the intersection causes further constriction of the sample fluid stream and re-orienting of the particles within the stream, which is caused by the asymmetric introduction of sheath fluid from both sides of the intersection, thus focusing the particles.

In some embodiments, the particles in the sample fluid are sperm cells, and because of their pancake-type or flattened teardrop shaped head, the sperm cells can re-orient themselves in a predetermined direction as they undergo asymmetric focusing—i.e., with their flat surfaces perpendicular to the direction of a light beam. Thus, the sperm cells develop a preference on their body orientation while passing through the two-step focusing process. Specifically, the sperm cells tend to be more stable with their flat bodies perpendicular to the direction of the compression. The sperm cells which start with random orientation achieve uniform orientation of approximately 90-99%, such as at least 91%, at least 92%, at least 93%, at least 94%, at least 95%, at least 96%, at least 97%, at least 98%, or at least 99%. The sperm cells not only make a single file formation at the center of the channel, but they also achieve a near uniform orientation. Thus, the particles introduced into sample input, which may be other types of cells or other materials as previously described, undergo asymmetric focusing, which orients and positions the particles in a single file formation, and in a more uniform orientation (depending on the type of particles), which allows for easier interrogation of the particles.

In addition to and/or in conjunction with the other embodiments recited herein, the present invention also provides a method of producing a fluid with gender-skewed sperm cells. The method may comprise providing any one of the asymmetrically focusing microfluidic devices described herein, flowing a semen fluid comprising sperm cells into the sample fluid channel and into the intersection, flowing a sheath fluid through the two sheath fluid channel structures, including through corresponding pluralities of tines, and into the intersection such that the sheath fluid causes laminar flow and compresses the semen fluid asymmetrically and from all sides, wherein the semen fluid becomes surrounded by sheath fluid and compressed into a thin stream, flowing the semen fluid and sheath fluids into the downstream flow channel, determining a chromosome type of the sperm cells in the semen fluid stream, wherein each sperm cell is either a Y-chromosome-bearing sperm cell or an X-chromosome-bearing sperm cell, and sorting Y-chromosome-bearing sperm cells from X-chromosome-bearing sperm cells or vice versa, thereby producing the fluid comprising gender-skewed sperm cells that are predominantly Y-chromosome-bearing or X-chromosome-bearing sperm cells.

In some embodiments, the chromosome type of the sperm cells may be determined using any one of the interrogation apparatus, devices, or methods described herein. In one embodiment, the microfluidic chip may further comprise an interrogation region downstream of the intersection. An interrogation apparatus coupled to the interrogation region is used to determine the chromosome type of the sperm cells and sort said sperm cells based on chromosome type. The interrogation apparatus may comprise a radiation source that illuminates and excites the sperm cells, and a response of the sperm cell is indicative of the chromosome type in the sperm cell. The response of the sperm cell may be detected by an optical sensor. In other embodiments, the interrogation apparatus may further comprise a laser source. The Y-chromosome-bearing sperm cells are sorted from the X-chromosome-bearing sperm cells or the X-chromosome-bearing sperm cells are sorted from the Y-chromosome-bearing sperm cells by laser ablation, which exposes the cells to the high intensity laser source that damages or kills cells that are determined to bear an X-chromosome or a Y-chromosome, depending on the characteristic or feature being enriched or selected for. In one embodiment, the gender-skewed sperm cells are comprised of at least 55% of Y-chromosome-bearing sperm cells. In another embodiment, the gender-skewed sperm cells are comprised of about 55%-99% of Y-chromosome-bearing sperm cells. In yet another embodiment, the gender-skewed sperm cells are comprised of at least 90%, at least 91%, at least 92%, at least 93%, at least 94%, at least 95%, or at least 96% of Y-chromosome-bearing sperm cells. In yet another embodiment, the gender-skewed sperm cells are comprised of at least 99% of Y-chromosome-bearing sperm cells. In one embodiment, the gender-skewed sperm cells are comprised of at least 55% of X-chromosome-bearing sperm cells. In another embodiment, the gender-skewed sperm cells are comprised of about 55%-99% of X-chromosome-bearing sperm cells. In yet another embodiment, the gender-skewed sperm cells are comprised of at least 90%, at least 91%, at least 92%, at least 93%, at least 94%, at least 95%, or at least 96% of X-chromosome-bearing sperm cells. In yet another embodiment, the gender-skewed sperm cells are comprised of at least 99% of X-chromosome-bearing sperm cells.

In one embodiment, the particles are detected in the interrogation chamber using a radiation source. The radiation source emits a light beam (which may be via an optical fiber) which is focused at the center of the channel widthwise. In one embodiment, the particles, such as sperm cells, are oriented by the asymmetric focusing and/or proximity to a surface such that the flat surfaces of the particles are facing toward the beam. In addition, all particles are preferably aligned in a substantially single file formation by focusing as they pass under a radiation source. As the particles pass under the radiation source and are acted upon by a light beam, the particles emit the fluorescence, which may be a Stoke shifted emission, which indicates the desired particles. For example, with respect to sperm cells, X chromosome cells fluoresce at a different intensity from Y chromosome cells; or cells carrying one trait may fluoresce in a different intensity or wavelength from cells carrying a different set of traits. In addition, the particles can be viewed for shape, size, or any other distinguishing indicators.

In one embodiment, interrogation of the sample containing particles (i.e., biological material), is accomplished by other methods. Overall, methods for interrogation may include direct visual imaging, such as with a camera, and may utilize direct bright-light imaging or fluorescent imaging; or, more sophisticated techniques may be used such as spectroscopy, transmission spectroscopy, spectral imaging, or scattering such as dynamic light scattering or diffusive wave spectroscopy. In some cases, the optical interrogation region may be used in conjunction with additives, such as chemicals which bind to or affect particles of the sample mixture or beads which are functionalized to bind and/or fluoresce in the presence of certain materials or diseases. These techniques may be used to measure cell concentrations, to detect disease, or to detect other parameters which characterize the particles.

However, in another embodiment, if fluorescence is not used, then polarized light back scattering methods may also be used. Using spectroscopic methods, the particles are interrogated and the spectrum of those particles which had positive results and fluoresced (i.e., those particles which reacted with a label) are identified for separation. In some embodiments, the particles may be identified based on the reaction or binding of the particles with additives or sheath or buffer fluids, or by using the natural fluorescence of the particles, or the fluorescence of a substance associated with the component, as an identity tag or background tag, or met a selected size, dimension, or surface feature, etc., are selected for separation. In one embodiment, upon completion of an assay, selection may be made, via computer and/or operator, of which particles to discard and which to collect.

Continuing with the embodiment of beam-induced fluorescence, the emitted light beam is then collected by the objective lens, and subsequently converted to an electronic signal by the optical sensor. The electronic signal is then digitized by an analog-digital converter (ADC) and sent to an electronic controller for signal processing. The electronic controller can be any electronic processer with adequate processing power, such as a DSP, a Micro Controller Unit (MCU), a Field Programmable Gate Array (FPGA), or even a Central Processing Unit (CPU). In one embodiment, the DSP-based controller monitors the electronic signal and may then trigger a sorting mechanism when a desired component is detected. In another embodiment, the FPGA-based controller monitors the electronic signal and then either communicates with the DSP controller or acts independently to trigger a sorting mechanism when a desired component is detected. In some other embodiments, the optical sensor may be a photomultiplier tube (PMT), an avalanche photodiode (APD), or a silicon photomultiplier (SiPM). In a preferred embodiment, the optical sensor may be an APD that detects the response of the sperm cell to interrogation.

In one embodiment of the sorting mechanism, the selected or desired particles in the interrogation chamber are isolated into a desired output channel using a piezoelectric actuator. In an exemplary embodiment, the electronic signal activates the driver to trigger the actuator at the moment when the target or selected component arrives at a cross-section point of jet channels and the micro-channel. This causes the actuator to contact a diaphragm and push it, compressing a jet chamber, and squeezing a strong jet of buffer or sheath fluids into the micro-channel, which pushes the selected or desired component into a desired output channel.

In some embodiments, the isolated particles are collected from their respective output channel for storing, further separation, or processing, such as cryopreservation. In some embodiments, the outputted particles may be characterized electronically, to detect concentrations of particles, pH measuring, cell counts, electrolyte concentration, etc.

In some embodiments, the microfluidic chip may be loaded on a chip cassette, which is mounted on chip holder. The chip holder is mounted to a translation stage to allow fine positioning of the holder. For instance, the microfluidic chip holder is configured to hold the microfluidic chip in a pre-determined position such that the interrogating light beam intercepts the fluid particles. In one embodiment, the microfluidic chip holder is made of a suitable material, such as aluminum alloy, or other suitable metallic/polymer material. A main body of the holder may be any suitable shape, but its configuration depends on the layout of the chip. In further embodiments, the main body of the holder is configured to receive and engage with external tubing for communicating fluids/samples to the microfluidic chip. A gasket of any desired shape, or O-rings, may be provided to maintain a tight seal between the microfluidic chip and the microfluidic chip holder. The gasket may be a single sheet or a plurality of particles, in any configuration, or material (i.e., rubber, silicone, etc.) as desired. In one embodiment, the gasket interfaces, or is bonded (using an epoxy) with a layer of the microfluidic chip. The gasket is configured to assist in sealing, as well as stabilizing or balancing the microfluidic chip in the microfluidic chip holder. The details of the cassette and holder and the mechanisms for attachment of the chip to the cassette and holder, are not described in any detail, as one of ordinary skill in the art would know that these devices are well-known and may be of any configuration to accommodate the microfluidic chip, as long as the objectives of the present invention are met.

In some embodiments, a pumping mechanism includes a system having a pressurized gas which provides pressure for pumping sample fluid mixture from reservoir (i.e., sample tube) into sample input of the chip. In other embodiments, a collapsible container having sheath or buffer fluid therein, is disposed in a pressurized vessel, and the pressurized gas pushes fluid such that fluid is delivered via tubing to the sheath or buffer input of the chip.

In one embodiment, a pressure regulator regulates the pressure of gas within the reservoir, and another pressure regulator regulates the pressure of gas within the vessel. A mass flow regulator controls the fluid pumped via tubing, respectively, into the sheath or buffer input. Thus, tubing is used in the initial loading of the fluids into the chip, and may be used throughout the chip to load a sample fluid into sample input.

In accordance with the present invention, any of the operations, steps, control options, etc. may be implemented by instructions that are stored on a computer-readable medium such as a memory, database, etc. Upon execution of the instructions stored on the computer-readable medium, for example, by a computing device or processor, the instructions can cause the computing device or processor to perform any of the operations, steps, control options, etc. described herein. In some embodiments the operations described in this specification may be implemented as operations performed by a data processing apparatus or processing circuit on data stored on one or more computer-readable storage devices or received from other sources. A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A program can be stored in a portion of a file that holds other programs or data, in a single file dedicated to the program in question, or in multiple coordinated files. A program can be deployed to be executed on one computer or on multiple computers interconnected by a communication network. Processing circuits suitable for the execution of a computer program include, by way of example, both general and special purpose microprocessors, and any one or more processors of any kind of digital computer.

In one embodiment, a user interface of the computer system includes a computer screen which displays the particles in a field of view acquired by a CCD camera or other imaging sensor over the microfluidic chip. In another embodiment, the computer controls any external devices such as pumps, if used, to pump any sample fluids, sheath or buffer fluids into the microfluidic chip, and also controls any heating devices which set the temperature of the fluids being inputted into the microfluidic chip.

It should be noted that the orientation of various elements may differ according to other illustrative embodiments, and that such variations are intended to be encompassed by the present disclosure. The construction and arrangements of the microfluidic chip, as shown in the various illustrative embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various illustrative embodiments without departing from the scope of the present disclosure.

While the invention has been described by reference to certain preferred embodiments, it should be understood that numerous changes could be made within the spirit and scope of the inventive concept described. Also, the systems and methods herein are not to be limited in scope by the specific embodiments described herein. It is fully contemplated that other various embodiments of and modifications to the systems and methods herein, in addition to those described herein, will become apparent to those of ordinary skill in the art from the foregoing description and accompanying drawings. Thus, such other embodiments and modifications are intended to fall within the scope of the following appended claims. Further, although the systems and methods herein have been described herein in the context of particular embodiments and implementations and applications and in particular environments, those of ordinary skill in the art will appreciate that their usefulness are not limited thereto and that the present invention can be beneficially applied in any number of ways and environments for any number of purposes. Accordingly, the claims set forth below should be construed in view of the full breadth and spirit of the systems and methods as disclosed herein.

What is claimed is:

1. A microfluidic channel structure comprising:
a sheath fluid channel structure originating upstream at a sheath fluid inlet, the sheath fluid channel structure comprising a first side channel structure and a second side channel structure;
a sample fluid channel originating at an upstream sample fluid inlet;
a flow channel disposed downstream of the sample fluid inlet and the sheath fluid inlet;
wherein the first side channel structure and the second side channel structure each comprise a plurality of tines extending from a main sheath fluid channel to the flow channel;
wherein the plurality of tines of both of the first channel structure and second channel structure intersect the flow channel at an intersection.

2. The microfluidic channel structure of claim 1, wherein the intersection of the plurality of tines with the flow channel is after a sample introduction location.

3. The microfluidic channel structure of claim 1, wherein the intersection of the plurality of tines with the flow channel is staggered.

4. The microfluidic channel structure of claim 1, wherein the intersection of the plurality of tines with the flow channel alternates between an intersection of a tine from the first side channel structure and an intersection of a tine from the second side channel structure.

5. (canceled)

6. (canceled)

7. The microfluidic channel structure of claim 1, wherein the plurality of tines intersects the flow channel at an angle of 45 degrees.

8. (canceled)

9. The microfluidic channel structure of claim 1, wherein a height of the tines is larger than a height of the flow channel.

10. The microfluidic channel structure of claim 1, wherein the intersection of each tine in the plurality of tines extends across the flow channel from a first side of the flow channel to a second side of the flow channel.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The microfluidic channel structure of claim 1, wherein the sample fluid channel intersects the flow channel upstream of the intersection of the sheath fluid channel structure and the flow channel.

23. The microfluidic channel structure of claim 1, wherein a height of the sample fluid channel is equal to a height of the flow channel.

24. (canceled)

25. (canceled)

26. (canceled)

27. (canceled)

28. (canceled)

29. (canceled)

30. (canceled)

31. (canceled)

32. The microfluidic channel structure of claim 1, wherein the flow channel comprises a detection region;
wherein a height of the detection region is less than a height of an upstream portion of the flow channel; and
wherein a width of the detection region is less than a width of an upstream portion of the flow channel.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The microfluidic channel structure of claim 1, wherein the intersection is configured to asymmetrically focus particles in a sample stream flowing through the flow channel.

48. The microfluidic channel structure of claim 1, wherein the intersection is configured to asymmetrically focus and orient particles in a sample stream flowing through the flow channel.

49. The microfluidic channel structure of claim 48, wherein the particles are sperm cells.

50. (canceled)

51. The microfluidic channel structure of claim 1, wherein the intersection is configured to asymmetrically focus a sample stream.

52. The microfluidic channel structure of claim 51, wherein the asymmetric focusing comprises flowing a first sheath fluid from a first tine in the plurality of tines of the first side channel structure into the flow channel to direct a sample stream in the flow channel towards a first side wall opposite of the first tine, and flowing a second sheath fluid from a second tine in the plurality of tines of the second side channel structure into the flow channel to direct the sample stream towards a second side wall opposite of the second tine.

53. The microfluidic channel structure of claim 51, wherein the plurality of tines of the first side channel structure is offset relative to the plurality of tines of the second side channel structure to provide for the asymmetric focusing of the sample stream.

54. The microfluidic channel structure of claim 51, wherein the asymmetric focusing comprises surrounding the sample stream with sheath fluid from substantially three directions to cause the sample stream to move towards a fourth direction, and subsequently surrounding the sample stream with sheath fluid from substantially three directions to cause the sample stream to move away from the fourth direction.

55. The microfluidic channel structure of claim 54, wherein the asymmetric focusing comprises repeatedly directing the sample stream towards and away from the fourth direction by repeatedly surrounding the sample stream with sheath fluid from at least three directions.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. The microfluidic channel structure of claim 1, wherein a sample fluid stream is positioned at a set height within the flow channel.

106. The microfluidic channel structure of claim 105, wherein a height of the plurality of tines of both the first and second microfluidic channel structure flows a sheath fluid stream into the flow channel to position the sample fluid stream at the set height within the flow channel.

107. (canceled)

108. (canceled)

109. (canceled)

110. (canceled)

111. (canceled)

112. The microfluidic channel structure of claim 105, wherein the set height is offset from a centerline of the flow channel.

113. (canceled)

114. (canceled)

115. (canceled)

116. (canceled)

117. (canceled)

118. (canceled)

119. (canceled)

120. (canceled)

121. (canceled)

122. (canceled)

123. A microfluidic chip comprising:
a first substrate layer and a second substrate layer;
a sheath fluid channel structure originating upstream at a sheath fluid inlet, the sheath fluid channel structure comprising a first side channel structure and a second side channel structure, wherein an upper portion of the first side channel structure and the second side channel structure are disposed in the first substrate layer, and wherein a lower portion of the first side channel structure and the section side channel structure are disposed in the second substrate layer;
a sample fluid channel originating at an upstream sample fluid inlet;
a flow channel disposed downstream of the sample fluid inlet and the sheath fluid inlet;
wherein the first side channel structure and the second side channel structure each comprise a plurality of tines extending from a main sheath fluid channel to the flow channel;
wherein the plurality of tines of both of the first channel structure and second channel structure intersect the flow channel at an intersection.

124. (canceled)

125. (canceled)

126. (canceled)

127. (canceled)

128. A method for focusing a sample fluid stream, the method comprising:
introducing a sheath fluid as a sheath fluid stream into a sheath fluid channel structure at a sheath fluid inlet;
diverting the sheath fluid stream into both a first side channel structure and a second side channel structure of the sheath fluid channel structure;
introducing a sample fluid as a sample fluid stream into a sample fluid channel at a sample fluid inlet;
flowing the sheath fluid stream into a plurality of tines of the first side channel structure;
flowing the sheath fluid stream into a plurality of tines of the second side channel structure;
flowing the sample fluid stream into a flow channel disposed downstream of the sample fluid inlet and the sheath fluid inlet;
intersecting the sample fluid stream in the flow channel at an intersection with the sheath fluid stream from the plurality of tines of the first side channel structure and from the plurality of tines of the second side channel structure; and
asymmetrically focusing the sample fluid stream by the sheath fluid stream at the intersection, wherein the sample fluid stream is focused into a smaller, narrower stream by asymmetric introduction of the sheath fluid stream from the plurality of tines of the first side channel structure and from the plurality of tines of the second side channel structure.

129. (canceled)

130. (canceled)

131. (canceled)

132. (canceled)

133. (canceled)
